(19) United States
(12) Patent Application Publication
(10) Pub. No.: US 2018/0028454 A1
(43) Pub. Date: Feb. 1, 2018

(54) PHARMACEUTICAL COMPOSITIONS AND METHODS OF USE THEREOF TO TREAT PANCREATIC ENZYME INSUFFICIENCY

(71) Applicant: AbbVie Inc., North Chicago, IL (US)

(72) Inventors: Jerry A. HALL, North Chicago, IL (US); Pedro Quintana DIAZ, North Chicago, IL (US); Shufang LlU, North Chicago, IL (US); Peter GREGORY, Hannover (DE); Beate JOHANNWILLE, Hannover (DE); Suntje SANDER-STRUCKMEIER, Hannover (DE); Hans-Friedrich KOCH, Hannover (DE)

(21) Appl. No.: 15/548,661

(22) PCT Filed: Feb. 4, 2016
(86) PCT No.: PCT/US2016/016610

§ 371 Date: Aug. 3, 2017

Related U.S. Application Data
(60) Provisional application No. 62/112,010, filed on Feb. 4, 2015.

(51) Int. Cl. A61K 9/48; A61K 9/20; A61K 38/46

(52) U.S. Cl. CPC A61K 9/4808; A61K 38/465; C12Y 301/01003; A61K 9/2018; A61K 9/2013; A61K 9/2031; A61K 9/4858; A61K 9/4866

(57) ABSTRACT

The present disclosure relates to pharmaceutical compositions comprising enzymes or enzyme mixtures having lipolytic and other optional other activities and methods of use thereof to treat exocrine pancreatic insufficiency.

### PRIORITY

This application claims the benefit of U.S. Provisional Patent Application No. 62/112,010, filed on Feb. 4, 2015, which is hereby incorporated by reference in its entirety.

### TECHNICAL FIELD

The present disclosure is directed to pharmaceutical compositions comprising enzymes or enzyme mixtures having lipolytic and optionally other enzyme activities, in particular pancrea-lipase/pancreatin. The pharmaceutical compositions provide improved lipolytic activity, in particular a stabilization of the lipase in the acidic pH range. These pharmaceutical compositions are suited for the treatment and/or prophylaxis of maldigestion, in particular maldigestion based on chronic exocrine pancreatic insufficiency, in mammals and humans.

### BACKGROUND OF THE INVENTION

Pancreatic enzyme products have long been used to treat exocrine pancreatic insufficiency, a condition associated with cystic fibrosis (CF), chronic pancreatitis, obstruction of the pancreas or common bile duct (such as from a neoplastic disease), surgical procedures such as pancreatectomy or gastrointestinal bypass surgery, as well as other diseases and disorders. The pancreas secretes digestive enzymes (lipases, proteases, and amylases) into the proximal duodenal lumen, where they facilitate the hydrolysis of macronutrients. Amylases and proteases are secreted by organs other than the pancreas, and these contribute to the digestion of carbohydrates and protein, there is relatively little lipase from sources other than the pancreas involved in digestion of lipids. Thus, patients with untreated exocrine pancreatic insufficiency typically have difficulty digesting fat and may suffer symptoms of maldigestion or malnutrition or both, with deficiencies of essential fatty acids and fat-soluble vitamins, weight loss, cramping. flatulence, bloating, and greasy, foul-smelling, loose stools (steatorrhea). For patients with CF, inadequate treatment may have serious consequences, as good nutritional status has been directly correlated with good lung function.

Pancreatic enzyme therapy treats and/or avoids malabsorption and facilitates growth and development in patients with CF due to pancreatic exocrine insufficiency (PEI). In patients with CF, mucus blocks the pancreatic duct in the pancreas as it does in the lungs. The pancreatic digestive enzymes are not secreted into the intestine, and thereby digestion of starch, fat, and protein is impaired. The lack of digestion results in steatorrhea, abdominal pain, and weight loss, among others.

Maldigestion in mammals and humans is usually based on a deficiency of digestive enzymes, in particular on a deficiency of endogenous lipase, but also of protease and/or amylase. If the pancreatic insufficiency is pathological, this may be congenital or acquired. Acquired chronic pancreatic insufficiency may, for example, be ascribed to alcoholism. Congenital pancreatic insufficiency may, for example, be ascribed to the congenital disease cystic fibrosis. The consequences of the deficiency of digestive enzymes may be severe symptoms of under-nutrition and malnutrition, which may be accompanied by increased susceptibility to secondary illnesses.

Substitution with similarly-acting exogenous digestive enzymes or mixtures of digestive enzymes (i.e., pancreatic enzyme therapy) has proved an effective treatment for a deficiency in endogenous digestive enzymes. Most frequently, pharmaceutical preparations containing porcine pancreatin are used for this purpose. Such mixtures of digestive enzymes obtained from the pig pancreas comprise lipases, amylases and proteases, and can be used effectively for pancreatic enzyme therapy (also known as “enzyme substitution therapy”) in humans owing to the great similarity of the enzymes and accompanying substances contained therein to the contents of human pancreatic juices. For example, processes are described in German patent applications DE 25 12746 and DE 42 03 315 by which pancreatin is obtained as a natural enzyme mixture by extraction from porcine pancreas and subsequently is converted in a known manner into the desired pharmaceutical form. The pancreatic enzymes are usually administered orally in the form of solid preparations.

Pancreatin/pancrelipase is commercially available, for example under the trade name Creon® in the form of granules, pellets or capsules with enteric-coated micropellets. Creon® pancrelipase capsules are a commercially available in various dosage forms: (1) a delayed-release dosage form comprising 3,000 USP units of lipase, 9,500 USP units of protease, 15,000 USP units of amylase; (2) a delayed-release dosage form comprising 6,000 USP units of lipase, 19,000 USP units of protease, 30,000 USP units of amylase; (3) a delayed-release dosage form comprising 12,000 USP units of lipase, 38,000 USP units of protease, 60,000 USP units of amylases; (4) a delayed-release dosage form comprising 24,000 USP units of lipase, 76,000 USP units of protease, 120,000 USP units; and (5) a delayed-release dosage form comprising 36,000 USP units of lipase, 114,000 USP units of protease, 180,000 USP units of amylase.

Creon® delayed-release capsules employ an enteric coating to ensure that the enzyme mixtures administered are not irreversibly denatured in the stomach by gastric acid and proteolytic enzymes, such as pepsin. Such a coating enables the intact enzyme mixtures to pass through the stomach as far as their point of action, the duodenum, where, due to the neutral to slightly alkaline conditions prevailing there, the protective enteric coating is broken down and the enzyme mixtures are released. Like the endogenous pancreatic enzymes of healthy humans, the orally supplied enzyme mixtures can then exert their enzymatic actions, in particular amylolytic, lipolytic and proteolytic actions. Such solid pancreatin formulations which can be coated with an enteric film are described e.g. in EP 0 021 129 A1.

EP 0583 726 A1 describes pancreatin micropellet cores coatable with an enteric film. The pancreatin cores have a pancreatin content of 65-85%, in particular 75-80% by weight, and a bulk density of 0.6 g/ml to 0.85 g/ml. They consist substantially of pancreatin, polyethylene glycol 4000 and low-viscosity paraffin, containing per 100 parts by weight pancreatin: 15-50, in particular 20-30, parts by weight polyethylene glycol 4000; and 1.5-5, in particular 2-3, parts by weight low-viscosity paraffin, and having a spherical to ellipsoid form, the sphere diameter or the minor axis being in the range of 0.7-1.4 mm, in particular 0.8-1.2 mm, and having a particle-size distribution in which at least 80% of the pancreatin micropellet cores have a ratio of minor axis to major axis in the range of 1:1 to 1:2.

Enteric coated pancreatin suffers from several drawbacks. First, patients with hyperacidic intestines may not benefit from enteric coated therapies due to the necessary alkaline environment to break down the enteric coating and release the digestive enzymes. Second, even though the enteric coating protects the enzyme mixture from degradation in the acidic stomach environment, the release of the enzyme mixture from the enteric coating is not immediate once the composition enters the duodenum. This delay can be significant because a significant portion of nutrients should be absorbed immediately, or as soon as possible, following passage through the pyloric sphincter and the lack of active enzyme(s) at that physiological location frustrates the timing and/or the level of absorption of macronutrients. Third, fibrosing colonopathy is a disease that arises in some patients with cystic fibrosis treated with enteric coated pancreatin. Patients with cystic fibrosis are at a higher risk of developing fibrosing colonopathy due to the high doses of enteric coated pancreatin needed to treat malabsorption.

For patients treated with oral pharmaceutical dosage forms having an enteric coating, it is known that at the point of action of the enzymes, in the duodenum, often only a small proportion of the lipase contained in the pharmaceutical formulation is active. In DE 36 42 853 A1, such enzyme deactivation is ascribed to insufficient neutralization of the gastric acid in the duodenum. Whereas in a healthy human the postprandial intraduodenal pH value is about 6, in patients with pancreatic insufficiency the postprandial intraduodenal pH can be about 4. At this pH value, the lipase contained in the pharmaceutical preparation has only one fifth of the efficacy that it would otherwise have at a pH value of 6.

Self-emulsifying pharmaceutical compositions in general are known from the prior art. For example, EP 0 670 715 describes a perorally administered composition which is suitable for forming a microemulsion in situ with the biological liquid of the organism and thus is said to improve the biological availability of an active substance. Such pharmaceutical compositions are known under the term SMEDDS (Self Microemulsifying Drug Delivery System) and consist in principle of a mixture of one or more active substances with a defined lipophilic phase, a defined surfactant and a defined co-surfactant, the properties of which are specified such that the end product is capable of forming a microemulsion on contact with a given volume of physiological liquid.

Furthermore, EP 1 058 540 B1 describes what is called a SMEDDS formulation in a particular pharmaceutical form, which is referred to as “pellet”. These pellets are composed of an active substance, in particular indomethacin, a binding agent which is suitable for improving the biological availability of the active substance, for example Gelucire® 44/14, and a diluent, for example lactose, in micronized form.

The object of the prior art self-emulsifying pharmaceutical compositions was to automatically form a microemulsion to increase the bioavailability of mostly lipophilic active substances in the SMEDDS formulation. The increase in bioavailability was a function of the micelle formation, which was said to permit better absorption of the active substance through the duodenal wall into the blood circulation.

EP 1 729 797 B1 and U.S. Pat. No. 8,802,087 describe pharmaceutical compositions of lipase-containing products for oral administration, including pancreatin-containing products, in which the pharmaceutical compositions provide improved lipolytic activity, in particular a stabilization of the lipase in the acidic pH range. These pharmaceutical compositions are characterized in that they contain a system which comprises at least one surfactant and one co-surfactant, and that they are self-emulsifiable on contact with a hydrophilic and a lipophilic phase. These pharmaceutical compositions are suited for the treatment and/or prophylaxis of maldigestion, in particular maldigestion based on chronic exocrine pancreatic insufficiency, in mammals and humans.

EP 0 826 375 A1 describes the use of lecithin as a stabilizing agent added to water-soluble pharmaceutical preparations of mixtures of digestive enzymes which contain protease/lipase mixtures, in particular pancreatin, and which are suitable for the preparation of aqueous solutions for continuous introduction into the gastrointestinal tract by means of probes. The lecithin is added to stabilize the mixtures of digestive enzymes against a decrease in the lipolytic activity under the influence of moisture.

Therefore, a medical need exists for better methods of treating exocrine pancreatic insufficiency which avoid one or more of the drawbacks associated with enteric coated pharmaceutical compositions.

### SUMMARY OF THE INVENTION

The present disclosure describes a method of treating exocrine pancreatic insufficiency in a subject in need of treatment, the method comprising administering a total daily dose of an enzyme or an enzyme mixture with at least lipolytic activity which exerts its action in the gastrointestinal tract. In some embodiments, the total daily dose, is about 240,000 lipase units or less, alternatively 122,000 lipase units or less, alternatively about 120,000 lipase units or less, alternatively 32,000 lipase units or less, alternatively about 30,000 lipase units or less, wherein the total daily dose is sufficient to treat exocrine pancreatic insufficiency, and the enzyme or enzyme mixture is in one or more pharmaceutical compositions which does not contain an enteric coating.

Further, the present disclosure discloses methods of treating diseases responsive to pancreatic enzyme replacement therapy comprising administering a dose of a pharmaceutical composition to a subject in need thereof; wherein the pharmaceutical composition comprises an enzyme or enzyme mixture with at least lipolytic activity and at least one surfactant; wherein the lipase dose of the pharmaceutical composition is less than about 300 U lipase/kg body weight/meal or less, alternatively 320 U lipase/kg body weight/meal.

The present disclosure also discloses methods of treating exocrine pancreatic insufficiency, comprising administering a dose of a pharmaceutical composition or oral pharmaceutical dosage form as described herein to a subject in need thereof; wherein the dose of the pharmaceutical composition or oral pharmaceutical dosage form comprises less than about 6,000 lipase units/kg body weight.

The present disclosure also describes pharmaceutical compositions. One pharmaceutical composition of the present disclosure comprises an enzyme or an enzyme mixture with at least lipolytic activity which exerts its action in the gastrointestinal tract, at least one surfactant, and at least one co-surfactant, wherein (1) the pharmaceutical composition is capable of releasing at least 85% of the lipolytic enzyme contained therein within one hour of contacting an in vivo pH equal or greater to 6.0; (2) the lipolytic enzyme is present in an amount equal to about 4,000 FIP units or about 30,000 FIP units, and/or (3) the pharmaceutical composition does not contain an enteric coating; and/or (4) the amount of the enzyme or enzyme mixture is greater than 60% by weight the total composition.

The present disclosure further discloses an oral pharmaceutical dosage form comprising an enzyme or an enzyme mixture with at least lipolytic activity which exerts its action in the gastrointestinal tract, and at least one surfactant, wherein the oral pharmaceutical dosage form releases at least 85% of the lipolytic enzyme contained therein within one hour of contacting an in vivo pH equal or greater to 6.0.

### DETAILED DESCRIPTION

The present disclosure describes pharmaceutical compositions comprising enzymes or enzyme mixtures with at least lipolytic activity and have improved lipolytic activity and, in particular, show a stabilization of the activity of the lipolytic enzyme in the acidic pH range. Additional pharmaceutical compositions and methods of making the same are disclosed in U.S. Application No. 62/075,467, which is hereby incorporated by reference in its entirety. These pharmaceutical compositions may be formulated into oral pharmaceutical dosage forms which exhibit stabilization of the activity of lipase in in vivo acidic environments, particularly in the stomach.

According to the present disclosure, pharmaceutical compositions comprising enzymes or enzyme mixtures with at least lipolytic activity; at least one surfactant; and at least one co-surfactant, and are characterized in that the at least one surfactant and the at least one co-surfactant can themselves be emulsified on contact with a hydrophilic phase and a lipophilic phase. Preferably, the hydrophilic phase used to form the final emulsion after ingestion of the oral pharmaceutical dosage form is supplied by the physiological fluid of the digestive milieu. In a further embodiment, the lipophilic phase used to form the final emulsion in the digestive tract after ingestion of the pharmaceutical composition, or an oral pharmaceutical dosage form thereof, is at least partially supplied by the lipids present in the food ingested.

Lipolytic activity is generally found in pancreatic enzymes or enzyme mixtures, such as porcine pancreatin. Lipolytic activity of an enzyme, enzyme mixture or pharmaceutical composition containing such an enzyme or mixture can be measured using established techniques. Likewise, proteolytic and amylolytic activities can be measured using known techniques. Assays for lipase, protease and amylase activity of porcine pancreatin have been published by the FIP (Federation Internationale Pharmaceutique) as well as the European Pharmacopoeia and the United States Pharmacopeia. Such assays determine the lipolytic activity of the enzyme or enzyme mixture in lipase units. 1 FIP-unit is equal to 1 Ph.Eur.-unit (European Pharmacopoeia). Appropriate enzyme standards can be procured from: International Commission on Pharmaceutical Enzymes, Centre for Standards, Harelbekestraat 72, B-9000 Ghent. As used herein, 1 “U lipase” or “lipase unit” is equal to 1 FIP-unit of lipase or 1 Ph.Eur.-unit of lipase.

The Cystic Fibrosis Foundation (CFF) has published Consensus Guidelines that contain recommended total daily doses of lipase units. Drug makers and physicians are encouraged to follow those Guidelines. By use of the present pharmaceutical compositions, it has unexpectedly been found that it is safe and effective to use doses of lipase which are much lower than the current CFF Consensus Guidelines.

Surprisingly, in certain embodiments, lipase-containing pharmaceutical compositions according to the present disclosure has improved lipolytic effectiveness and a lipolytic activity which is stabilized in the acidic pH range. The use of the disclosed pharmaceutical compositions display equal or greater efficacy compared to similar pharmaceutical compositions with enteric coatings, such as those described in EP 0 583 726 A1 and others. In certain embodiments, the pharmaceutical compositions of the present disclosure comprise enzymes or enzyme mixtures with at least lipolytic activity, at least one surfactant, at least one co-surfactant and optionally a lipophilic phase, wherein a reduction in the lipolytic activity during passage through the stomach is significantly less than with conventional pharmaceutical compositions due to the present pharmaceutical composition or oral pharmaceutical dosage form being stabilized in the acidic pH range of the stomach compared with conventional formulations.

The fact that the use of such enteric-coated polymer films and softeners which are otherwise necessary for film-coating various dosage forms (granules, pellets, mini-tablets, tablets etc.) can be dispensed with in the preparation of the present lipase-containing pharmaceutical composition yields further advantages. For example, the safety profile of methods of treating exocrine pancreatic insufficiency using the pharmaceutical composition of the present disclosure is improved by omitting the enteric polymer films and softeners because improved efficacy is observed in the present methods using compositions without enteric coating. In particular, the risks of developing or exacerbating fibrosing colonopathy is greatly reduced in methods employing the pharmaceutical compositions or oral pharmaceutical dosage forms of the present disclosure compared to methods employing conventional compositions or oral pharmaceutical dosage forms. Furthermore, the proportions of the amount of film-coating material in conventional dosage forms comprise an enteric coating that is approximately 20-30% of the entire weight of the dosage form. Dispensing with these materials makes the amount of oral pharmaceutical dosage forms of the present disclosure smaller in size, which results in improved patient compliance in the methods of treatment described herein.

The possibility of dispensing with enteric coating of the enzymes or enzyme mixtures furthermore has the advantage that thorough mixing of the pharmaceutical composition, more particularly the enzyme or enzyme mixtures, with the chyme can take place as early as in the stomach. Thereupon, an emulsion and/or microemulsion is formed with enlarged surface, on which the lipolytic enzyme contained in the pharmaceutical composition is distributed such that it is given increased opportunity for contacting and breaking down the triglycerides found in the chyme. The formation of emulsions and microemulsions of the enzyme or enzyme mixtures within the stomach is further intensified by the lipolytic breakdown of the triglycerides to form di- and monoglycerides and free fatty acids. Thus, increased opportunity for lipolytic activity results from the intensified breakdown of the triglycerides, resulting in a higher concentration of free fatty acids from the food. This in turn results in better fat absorption in the duodenum. In vitro, an increase in the lipolytic effectiveness (such as by increasing absorption of fats) by about 10% compared with conventional lipase-containing pharmaceutical preparations was determined for certain embodiments of the pharmaceutical compositions of the present disclosure. In one embodiment, use of the pharmaceutical composition or oral pharmaceutical dosage form of the present disclosure results in a reduction in the dose of lipase compared to use of conventional pharmaceutical compositions or oral pharmaceutical dosage forms. For example, the amount or dose of lipase administered to a subject using the present pharmaceutical compositions or oral pharmaceutical dosage forms may be reduced at least 40%, 50%, 60%, 65%, 70%, 75%, 80%, or 90% relative to conventional lipolytic treatments.

The pharmaceutical compositions and oral pharmaceutical dosage forms of the present disclosure exhibit stabilization of the lipolytic activity in the stomach as well as in the duodenum; additionally, owing to the formation of an emulsion and/or microemulsion, the lipolytic activity is increased. The (micro)emulsion already produced independently in the stomach results in better activation of the lipase contained in the pharmaceutical composition or oral pharmaceutical dosage form of the present disclosure.

In contrast to prior pharmaceutical compositions designed to deliver an active agent through the duodenal wall into the bloodstream, the pharmaceutical compositions and oral pharmaceutical dosage forms of the present disclosure do not contain any lipophilic active agents intended to be absorbed into the bloodstream. Instead, the present pharmaceutical compositions, oral pharmaceutical dosage forms, and methods of use thereof are designed to deliver active enzymes or enzyme mixtures with at least lipolytic activity to the gastrointestinal tract, wherein the active enzymes act on macronutrients that will be absorbed through the duodenal wall. The self-emulsifiable pharmaceutical compositions and oral pharmaceutical dosage forms of the present disclosure result in a surprising increase of the lipolytic activity and an improved stability of the lipase in the acidic pH range. Such pharmaceutical compositions of lipase-containing enzyme products which are self-emulsifiable on contact with a hydrophilic phase is due at least in part to the at least one surfactant and at least one co-surfactant of the pharmaceutical compositions or oral pharmaceutical dosage forms of the present disclosure.

Subramanian and Wasan describe an assay in which they demonstrate that the substance Gelucire® 44/14 in vitro has an inhibiting effect on pancreatic lipase activity [Subramanian R. & Wasan K. M. (2003) “Effect of lipid excipients on in vitro pancreatic lipase activity” Drug Dev. Ind. Pharm. 29(8): 885-90]. In this assay, a particular lipid-containing assay buffer is mixed with separate solutions of Gelucine® 44/14 surfactant, pancreatic lipase and co-lipase to form several samples, and the influence of Gelucire® surfactant on the lipase activity is measured. Since the measured lipase activity decreases, the authors conclude that Gelucire® surfactant and similar lipidic additions to pharmaceutical formulations can have an adverse effect on the in vitro activity of the pancreatic lipase. In contrast, the present disclosure shows that self-emulsifiable pharmaceutical compositions consisting of lipase-containing enzyme mixtures and surfactants such as for example Gelucire® 44/14 surfactant result in an increase in the lipolytic activity contained in the present pharmaceutical compositions or oral pharmaceutical dosage forms.

In addition to the preserved lipolytic activity of the enzymes in the present pharmaceutical compositions and oral pharmaceutical dosage forms, it is contemplated that the present pharmaceutical compositions, oral pharmaceutical dosage forms, and methods of use thereof will result in preserved activity of one or more proteases and/or one or more amylases compared to conventional pharmaceutical compositions or oral pharmaceutical dosage forms. The preservation of enzymatic activity is due to the stabilization of the present pharmaceutical composition or oral pharmaceutical dosage form in acidic environments coupled to the delivery of active enzymes at the desired location within the duodenum. Consequently, the amount or dose of protease in the present pharmaceutical compositions or oral pharmaceutical dosage forms may be reduced at least 40%, 50%, 60%, 65%, 70%, 75%, 80%, or 90% compared to conventional pharmaceutical compositions or oral pharmaceutical dosage forms. Similarly, the amount or dose of the amylase in the present pharmaceutical compositions or oral pharmaceutical dosage forms for use in the present methods may be reduced at least 10%, 20%, 30%, 40%, 50%, or 60% compared to conventional pharmaceutical compositions or oral pharmaceutical dosage forms.

Experimental evidence demonstrates that methods using the present pharmaceutical compositions and oral pharmaceutical dosage forms produce in in vivo effects earlier than methods using conventional therapies. For example, administration of the present pharmaceutical composition resulted in maximal plasma concentration (max) of para-aminobenzoic acid (PABA) within 1 hour of administration. In contrast, administration of the conventional Creon® capsules resulted in a maximal plasma concentration (max) of PABA after 2-3 hours after administration of Creon® capsules. PABA is used as a marker of pancreatic function. In some embodiments, the present pharmaceutical compositions and oral pharmaceutical dosage forms are capable of releasing a total percentage of lipase within a certain period of time after coming into contact with a certain pH, for example, in the duodenum, as set forth in Table 1.

TABLE 1

| Percent of Lipase Released | Time (min) | pH |
|---|---|---|
| 95 | 60 | ≧6 |
| 95 | 60 | ≧5 |
| 95 | 60 | ≧4 |
| 95 | 50 | ≧6 |
| 95 | 50 | ≧5 |
| 95 | 50 | ≧4 |
| 95 | 40 | ≧6 |
| 95 | 40 | ≧5 |
| 95 | 40 | ≧4 |
| 95 | 30 | ≧6 |
| 95 | 30 | ≧5 |
| 95 | 30 | ≧4 |
| 95 | 20 | ≧6 |
| 95 | 20 | ≧5 |
| 95 | 20 | ≧4 |
| ≧90 | 60 | ≧6 |
| ≧90 | 60 | ≧5 |
| ≧90 | 60 | ≧4 |
| ≧90 | 50 | ≧6 |
| ≧90 | 50 | ≧5 |
| ≧90 | 50 | ≧4 |
| ≧90 | 40 | ≧6 |
| ≧90 | 40 | ≧5 |
| ≧90 | 40 | ≧4 |
| ≧90 | 30 | ≧6 |
| ≧90 | 30 | ≧5 |
| ≧90 | 30 | ≧4 |
| ≧90 | 20 | ≧6 |
| ≧90 | 20 | ≧5 |
| ≧90 | 20 | ≧4 |
| ≧85 | 60 | ≧6 |
| ≧85 | 60 | ≧5 |
| ≧85 | 60 | ≧4 |
| ≧85 | 50 | ≧6 |
| ≧85 | 50 | ≧5 |
| ≧85 | 50 | ≧4 |
| ≧85 | 40 | ≧6 |
| ≧85 | 40 | ≧5 |
| ≧85 | 40 | ≧4 |
| ≧85 | 30 | ≧6 |
| ≧85 | 30 | ≧5 |
| ≧85 | 30 | ≧4 |
| ≧85 | 20 | ≧6 |
| ≧85 | 20 | ≧5 |
| ≧85 | 20 | ≧4 |
| ≧80 | 60 | ≧6 |
| ≧80 | 60 | ≧5 |
| ≧80 | 60 | ≧4 |
| ≧80 | 50 | ≧6 |
| ≧80 | 50 | ≧5 |
| ≧80 | 50 | ≧4 |
| ≧80 | 40 | ≧6 |
| ≧80 | 40 | ≧5 |
| ≧80 | 40 | ≧4 |
| ≧80 | 30 | ≧6 |
| ≧80 | 30 | ≧5 |
| ≧80 | 30 | ≧4 |
| ≧80 | 20 | ≧6 |
| ≧80 | 20 | ≧5 |
| ≧80 | 20 | ≧4 |
| ≧75 | 60 | ≧6 |
| ≧75 | 60 | ≧5 |
| ≧75 | 60 | ≧4 |
| ≧75 | 50 | ≧6 |
| ≧75 | 50 | ≧5 |
| ≧75 | 50 | ≧4 |
| ≧75 | 40 | ≧6 |
| ≧75 | 40 | ≧5 |
| ≧75 | 40 | ≧4 |
| ≧75 | 30 | ≧6 |
| ≧75 | 30 | ≧5 |
| ≧75 | 30 | ≧4 |
| ≧75 | 20 | ≧6 |
| ≧75 | 20 | ≧5 |
| ≧75 | 20 | ≧4 |

For all patient populations, the starting dose of conventional Creon® capsules is generally the lowest recommended dose and then increased if necessary based on based on clinical symptoms, the degree of steatorrhea present, and/or the fat content of the patient's particular diet. The dosing regimens using conventional Creon® capsules vary according to the age of the patient. The present pharmaceutical compositions or dosage forms have the same dosing recommendations: starting dose and then increased if necessary; however, in the present methods, the present pharmaceutical compositions allow the enzymatic doses, particularly lipase, to be greatly reduced relative to conventional Creon® capsules while yielding equivalent or superior lipolytic effect. The lower lipase dose of the present pharmaceutical composition in the present methods is a significant improvement over conventional Creon® capsules for a number of reasons. First, treatment-naïve patients may experience a lower risk of treatment-related side effects due to the lower active ingredient amount while efficacy is not compromised with the lower dose of the present pharmaceutical composition. Second, patients who received conventional Creon® capsules and experienced treatment-related side effects that limited the maximum tolerated dose may benefit from the lower dose of lipase in the present method, which has equivalent or better efficacy. Third, patients who received conventional Creon® capsules and switch to the present pharmaceutical composition may benefit from a lower present and future risk of treatment-related side effects due to the lower lipase dose. Fourth, the lower lipase dose of the present pharmaceutical composition also translates to smaller intra-range increases in lipase dose, which may translate into lesser treatment-related side effects. Fifth, patients who are receiving pharmaceutical treatments other than lipolytic enzyme therapy (e.g., antibiotics) may benefit from the lower lipase dose of the present pharmaceutical composition. Sixth, patients suffering from gout, renal impairment, or hyperuricemia may benefit from the lower lipase dose of the present pharmaceutical composition due to the risk of hyperuricemia associated with conventional Creon® capsules. Seventh, patients who may have an allergic reaction or hypersensitivity to the higher doses of the conventional Creon® capsules may avoid or lessen the allergic reaction by using the lower lipase dose of the present pharmaceutical compositions. Eighth, the risk of viral exposure may be decreased with the use of the lower lipase dose of the present pharmaceutical composition compared to conventional Creon® capsules. Ninth, the risk of developing fibrosing colonopathy associated with high doses of pancreatic enzyme products may be decreased through the use of the lower lipase dose of the present pharmaceutical composition relative to the conventional therapy Creon® capsules. Other benefits will be apparent to those skilled in the art.

Dosing Regimens for Infants.

Conventional Creon® capsules are administered to infants (up to 12 months) at a dose of 2,000 to 4,000 U lipase per 120 mL of formula or per breast-feeding. One breast-feeding or 120 mL of formula is considered a meal for infants. Generally, the lowest dose is administered to the infant and then increased, if necessary, based on clinical symptoms or the degree of steatorrhea present.

The dosage range for the present pharmaceutical composition in the present methods for infants (up to 12 months) ranges from about 500 to about 1250 U lipase/meal; alternatively, about 667 to about 1250 U lipase/meal; alternatively, about 1000 to about 2500 U lipase/meal; alternatively, about 1333 to about 3333 U lipase/meal; or alternatively, about 1538 to about 3846 U lipase/meal. In some embodiments, the method comprises administering the pharmaceutical composition at a dose no higher than the upper limit of the foregoing ranges.

The maximum daily lipase dose of the present pharmaceutical composition in the present methods for infants is significantly reduced compared to conventional Creon® capsules. The maximum lipase dose for conventional Creon® capsules is 2,500 U lipase/kg body weight/meal or 10,000 U lipase/kg body weight/day. Depending on factors within the purview of a health care provider, the present pharmaceutical composition may be administered to infants based on a per meal or per day dosing regimen. For example, it may be more prudent to use a per day dosing regimen for an infant who feeds significantly more or less than a normal infant feeding schedule. In embodiments, the maximum lipase dose of the present pharmaceutical composition for infants should not exceed about 625 U lipase/kg body weight/meal; alternatively, about 833 U lipase/kg body weight/meal; alternatively, about 1,250 U lipase/kg body weight/meal; alternatively, about 1,667 U lipase/kg body weight/meal; or alternatively, about 1,923 U lipase/kg body weight/meal. In other embodiments, the maximum lipase dose of the present pharmaceutical composition for infants should not exceed about 2,500 U lipase/kg body weight/day; alternatively, about 3,333 U lipase/kg body weight/day; alternatively, about 5,000 U lipase/kg body weight/day; alternatively, about 6,667 U lipase/kg body weight/day; or alternatively, about 7,692 U lipase/kg body weight/day. In other embodiments, the total lipase daily lipase dose may be equal to or lesser than the CFF guidelines.

In some embodiments, the infant's meal is consumed concurrently with the present pharmaceutical composition. In preferred embodiments, the infant's meal is consumed immediately after the present pharmaceutical composition is administered.

The present pharmaceutical composition may be administered orally to infants. In embodiments, the present pharmaceutical composition may be added to a palatable vehicle (e.g., applesauce). Preferably, the palatable vehicle is slightly acidic with a pH of 4 or less; alternatively, a pH of 5 or less; alternatively, a pH of 6 or less; or alternatively, a pH less than 7. To maximize efficacy, the present pharmaceutical composition should not be added directly to the formula or breast milk, and care must be taken to avoid excessive time in the oral cavity or mastication.

The doses of the present compositions for the treatment of infant may be used to treat exocrine pancreatic insufficiency. In embodiments, the pancreatic exocrine insufficiency may be affiliated with, due to, or caused by cystic fibrosis, chronic pancreatitis, or pancreatectomy.

Dosing Regimens for Children Older than 12 Months and Younger than 4 Years.

The lowest recommended dose of conventional therapy Creon® capsules for children older than 12 months and younger than 4 years old is 1,000 U lipase/kg body weight/meal resulting in a dose of 4,000 total U lipase/kg body weight/day (i.e., 1,000 U lipase/kg body weight×3 meals and 500 U lipase/kg body weight×2 snacks). The dose of conventional Creon® capsules for children older than 12 months and younger than 4 years old may be increased up to 2,500 U lipase/kg body weight/meal, if necessary. Generally, for children older than 12 months and younger than 4 years, conventional Creon® capsules begins with lowest recommended dose and then is increased if necessary based on based on clinical symptoms, the degree of steatorrhea present, and/or the fat content of the child's particular diet.

The first dose of the present pharmaceutical composition or dosage form for the treatment of children older than 12 months and younger than 4 years old comprises about 250 U lipase/kg body weight/meal; alternatively, about 333 U lipase/kg body weight/meal; alternatively, about 500 U lipase/kg body weight/meal; alternatively, about 667 U lipase/kg body weight/meal; or alternatively, about 769 U lipase/kg body weight/meal. Subsequent higher doses of the present pharmaceutical composition or dosage form may be administered to alleviate clinical symptoms, if necessary, but should not exceed a maximum dose. Subsequent higher doses may be about 625 U lipase/kg body weight/meal; alternatively, about 833 U lipase/kg body weight/meal; alternatively, about 1,250 U lipase/kg body weight/meal; alternatively, about 1,667 U lipase/kg body weight/meal; or alternatively, about 1,923 U lipase/kg body weight/meal. A subsequent higher dose may comprise a second dose; a third dose; a fourth dose; or a fifth dose. The first dose may be the lowest recommended dose.

The dosing schedule for the present pharmaceutical composition or dosage form may allow for administration to children older than 12 months and younger than 4 years old concurrently with meals and snacks. In embodiments, the dose is administered before a meal or snack. In further embodiments, the dose is administered after a meal or snack. In still further embodiments, the dose is administered before, during, and after a snack. The dosing schedule of the present pharmaceutical composition or dosage form for treatment of children older than 12 months and younger than 4 years old may follow one of the dosing schedules described in Dominguez-Muñoz J E, Iglesias-García J, Iglesias-Rey M, et al. “Effect of the administration schedule on the therapeutic efficacy of oral pancreatic enzyme supplements in patients with exocrine pancreatic insufficiency: a randomized, three-way crossover study.” Aliment Pharmacol Ther. 2005; 21:993-1000, which is hereby incorporated by reference in its entirety.

The maximum dose of the conventional therapy Creon® is less than or equal to 10,000 U lipase/kg body weight/day or 4,000 U lipase/g fat ingested/day. In embodiments, the maximum dose of the present pharmaceutical composition or dosage form for the treatment of children older than 12 months and younger than 4 years old comprises about 2,500 U lipase/kg body weight/day; alternatively, about 3,333 U lipase/kg body weight/day; alternatively, about 5,000 U lipase/kg body weight/day; alternatively, about 6,667 U lipase/kg body weight/day; or alternatively, about 7,692 U lipase/kg body weight/day. In other embodiments, maximum dose of the present pharmaceutical composition or dosage form for the treatment of children older than 12 months and younger than 4 years old comprises about 1,000 U lipase/g fat ingested/day; alternatively, about 1,333 U lipase/g fat ingested/day; alternatively, about 2,000 U lipase/g fat ingested/day; alternatively, about 2,667 U lipase/g fat ingested/day; or alternatively, about 3077 U lipase/g fat ingested/day. In other embodiments, the total lipase daily lipase dose may be equal to or lesser than the CFF guidelines.

The doses of the present compositions for the treatment of children older than 12 months and younger than 4 years old may be used to treat pancreatic exocrine insufficiency. In embodiments, the pancreatic exocrine insufficiency may be affiliated with, due to, or caused by cystic fibrosis, chronic pancreatitis, or pancreatectomy.

Dosing Regimens for Adults and Children Aged 4+.

The lowest recommended dose of conventional therapy Creon® for adults and children aged four and above is 500 U lipase/kg body weight/meal resulting in a 2,000 total lipase units/kg body weight per day (i.e., 500 U lipase/kg body weight×3 meals and 250 U lipase/kg body weight×2 snacks). It is contemplated that the use of the present pharmaceutical composition or oral pharmaceutical dosage form would similarly be initiated at a low dose and then, optionally, escalated if necessary based on clinical symptoms, the degree of steatorrhea present, and/or the content of the patient's diet. Similarly, the lipase dose of the present pharmaceutical composition or oral dosage form may be approximately 2× the lipase dose of a snack. It is contemplated the first dose of the present pharmaceutical composition or oral pharmaceutical dosage form and methods of use thereof is significantly reduced compared to conventional therapies. In embodiments, the first dose of the present pharmaceutical composition or oral pharmaceutical dosage form for adults and children aged four and above comprises about 50 U lipase/kg body weight/meal; alternatively, about 75 lipase U/kg body weight/meal; alternatively, 100 U lipase/kg body weight/meal; alternatively, about 125 U lipase/kg body weight/meal; alternatively, about 150 U lipase/kg body weight/meal; alternatively, about 167 U lipase/kg body weight/meal; alternatively, about 200 U lipase/kg body weight/meal; alternatively, about 250 U lipase/kg body weight/meal; alternatively, about 300 U lipase/kg body weight/meal; alternatively, about 333 U lipase/kg body weight/meal; alternatively, about 350 U lipase/kg body weight/meal; alternatively, about 385 U lipase/kg body weight/meal; alternatively, less than 400 U lipase/kg body weight/meal; or alternatively, less than 500 U lipase/kg body weight/meal.

Subsequent higher doses of the present pharmaceutical composition or dosage form may be administered to alleviate clinical symptoms, if necessary, but should not exceed a maximum dose. Again, the higher lipase dose may be administered with every meal and a lower lipase dose may be administered with each snack. In embodiments, the higher lipase dose for adults and children aged four and above comprises about 250 lipase U/kg body weight/meal; alternatively, about 333 lipase U/kg body weight/meal; alternatively, about 400 lipase U/kg body weight/meal; alternatively, 500 lipase U/kg body weight/meal; alternatively, about 600 lipase U/kg body weight/meal; alternatively, about 667 lipase U/kg body weight/meal; alternatively, about 769 lipase U/kg body weight/meal; alternatively, about 800 lipase U/kg body weight/meal; alternatively, about 1,000 lipase U/kg body weight/meal; alternatively, about 1,200 lipase U/kg body weight/meal; alternatively, about 1,400 lipase U/kg body weight/meal; or, alternatively, less than 2,000 lipase U/kg body weight/meal. If disease symptoms persist despite the administration of the higher lipase dose, further higher lipase dose(s) may be administered until the desired clinical effect is observed. A subsequent higher dose may comprise a second dose; a third dose; a fourth dose; or a fifth dose. The first dose may be the lowest recommended dose.

The dosing schedule for the present pharmaceutical composition or dosage form may allow for administration to adults and children aged four and above concurrently with meals and snacks. In embodiments, the dose is administered before a meal or snack. In further embodiments, the dose is administered after a meal or snack. In still further embodiments, the dose is administered before, during, and after a snack. The dosing schedule of the present pharmaceutical composition or dosage form for treatment of adults and children aged four and above may follow one of the dosing schedules described in Dominguez-Muñoz J E, Iglesias-García J, Iglesias-Rey M, et al. “Effect of the administration schedule on the therapeutic efficacy of oral pancreatic enzyme supplements in patients with exocrine pancreatic insufficiency: a randomized, three-way crossover study.” Aliment Pharmacol Ther. 2005; 21:993-1000.

Administration of the present pharmaceutical composition or oral pharmaceutical dosage forms may not exceed the maximum lipase dose and/or the maximum daily lipase daily dose. The maximum daily lipase dose of the present pharmaceutical composition and oral pharmaceutical dosage forms is significantly reduced compared to conventional therapies. In embodiments, the maximum lipase dose for adults and children aged four and above comprises about 200 U lipase/kg body weight/meal; alternatively, about 300 U lipase/kg body weight/meal; alternatively, about 400 U lipase/kg body weight/meal; alternatively, about 500 U lipase/kg body weight/meal; alternatively, about 600 U lipase/kg body weight/meal; alternatively, about 800 U lipase/kg body weight/meal; alternatively, about 1,000 U lipase/kg body weight/meal; alternatively, about 1,200 U lipase/kg body weight/meal; alternatively, about 1,600 U lipase/kg body weight/meal; alternatively, about 3,200 U lipase/kg body weight/meal; alternatively, about 4000 U lipase/kg body weight/meal; alternatively, about 4,800 U lipase/kg body weight/meal; alternatively, about 5,600 U lipase/kg body weight/meal; or, alternatively, less than 8,000 U lipase/kg body weight/meal. In other embodiments, the maximum lipase dose for children over the age of four and adults comprises about 2,500 U lipase/kg body weight/day; alternatively, about 3,333 U lipase/kg body weight/day; alternatively, about 5,000 U lipase/kg bodyweight/day; alternatively, about 6667 U lipase/kg body weight/day; alternatively, about 7,692 U lipase/kg body weight/day; or alternatively, less than 10,000 U lipase/kg body weight/day. In other embodiments, the maximum lipase daily dose for adults and children aged four and above comprises about 800 U lipase/gram of fat/day; alternatively, about 1,000 U lipase/gram of fat/day; alternatively, about 1,200 U lipase/gram of fat/day; alternatively, about 1.333 U lipase/gram of fat/day; alternatively, about 1,600 U lipase/gram of fat/day; alternatively, about 1800 U lipase/gram of fat/day; alternatively, about 2,000 U lipase/gram of fat/day; alternatively, about 2,200 U lipase/gram of fat/day; alternatively, about 2,400 U lipase/gram of fat/day; alternatively, about 2,667 lipase units/gram of fat/day; alternatively, about 2800 U lipase/gram of fat/day; alternatively, about 3,077 U lipase/gram of fat/day; or, alternatively, less than 4,000 U lipase/gram of fat/day. In further embodiments, the maximum lipase daily dose for adults and children aged four and above comprises about 30,000 U lipase/day; alternatively, about 80,000 U lipase/day; alternatively, about 120,000 U lipase/day; alternatively, about 180,000 U lipase/day; alternatively, about 240,000 U lipase/day; or, alternatively, less than 400,000 U lipase/day. In other embodiments, the total lipase daily lipase dose may be equal to or lesser than the CFF guidelines.

The doses of the present compositions for the treatment of adults and children aged four and above may be used to treat pancreatic exocrine insufficiency. In embodiments, the pancreatic exocrine insufficiency may be affiliated with, due to, or caused by cystic fibrosis, chronic pancreatitis, or pancreatectomy.

In embodiments, the total dose by weight (units per kg) of the present pharmaceutical compositions or oral pharmaceutical dosage forms may be lower than conventional therapies. Without being bound by any particular theory, the reduction in dose may be in part attributable to the increased efficacy or protein efficiency associated with the use of the present pharmaceutical compositions or oral pharmaceutical dosage forms.

In embodiments, the oral pharmaceutical dosage forms may be capsules comprising 6,000, 12,000, 24,000, or 36,000 lipase units per capsule. In other embodiments designed for pediatric treatments, the oral pharmaceutical dosage forms may be capsules comprising, 3,000, 4,000, 6,000, or 8,000 lipase units per capsule.

The following terms are defined for use in interpreting the present disclosure.

“Treat” or “treating” in the present disclosure includes alleviating symptoms, enhancing metabolism and or absorption of nutrients, arresting, slowing, retarding, or stabilizing progression of a condition or physiological or morphological market thereof, and/or improving a clinical outcome, for example as measured by quality of life, incidence or severity of adverse events associated with exocrine pancreatic insufficiency; diseases that result in malabsorption, including cystic fibrosis; or diseases that require pancreatic enzyme therapy.

The “hydrophilic-lipophilic balance” value (the “HLB” value) is an empirical parameter commonly used to characterize the relative hydrophilicity and lipophilicity of non-ionic amphiphilic compounds is the hydrophilic-lipophilic balance. Surfactants or co-surfactants with lower HLB values are more lipophilic, and have greater solubility in oils, whereas surfactants or co-surfactants with higher HLB values are more hydrophilic, and have greater solubility in aqueous solutions. It should be kept in mind, that for anionic, cationic, or zwitterionic compounds the HLB scale is not generally applicable.

Generally, the HLB value of a surfactant or co-surfactants is a practical guide used to enable formulation of industrial, pharmaceutical and cosmetic emulsions. However, for many important surfactants, including several polyethoxylated surfactants, it has been reported that HLB values can differ by as much as about 8 HLB units, depending upon the empirical method chosen to determine the HLB value [Schott, J. Pharm. Sciences, 79(1), 87-88 (1990)]. Likewise, for certain polypropylene oxide containing block copolymers (poloxamers), the HLB values may not accurately reflect the true physical chemical nature of the compounds. Finally, commercial surfactant and/or co-surfactant products are generally not pure compounds, but are often complex mixtures of compounds, and the HLB value reported for a particular compound may more accurately be characteristic of the commercial product of which the compound is a major component. Different commercial products having the same primary surfactant and/or co-surfactant component can, and typically do, have different HLB values. In addition, a certain amount of lot-to-lot variability is expected even for a single commercial surfactant and/or co-surfactant product.

A surfactant in the context of the present disclosure is a chemical compound comprising two groups, the first being hydrophilic and/or polar or ionic and having a high affinity for water, and the second containing an aliphatic chain of greater or lesser length and being hydrophobic (lipophilic); i.e., a surfactant compound must be amphiphilic. These chemical compounds are intended to cause the formation and stabilisation of oil-in-water emulsions. Surfactants with lower HLB values are more lipophilic, and have greater solubility in oils, whereas surfactants with higher HLB values are more hydrophilic, and have greater solubility in aqueous solutions. Suitable surfactants in the context of the present disclosure have an HLB value above 6 and below 18, preferably above 8 and below 16. Surfactants can be any surfactant suitable for use in pharmaceutical compositions. Suitable surfactants can be anionic, cationic, zwitterionic or non-ionic. Such surfactants can be grouped into some general chemical classes as explained below. It should be emphasized that the disclosure is not limited to the surfactants indicated herein, which show representative, but not exclusive, lists of available surfactants.

PEG-Fatty Acid Monoester Surfactants:

Although polyethylene glycol (PEG) itself does not function as a surfactant, a variety of PEG-fatty acid esters have useful surfactant properties. Particulary preferred are PEG-fatty acid monoesters with aliphatic C6-C22 carboxylic acids, whereby the polyethylene glycol comprises 6 to 60 ethylene oxide units per molecule. Examples of polyethoxylated fatty acid monoester surfactants commercially available are: PEG-4 laurate, PEG-4 oleate, PEG-4 stearate, PEG-5 stearate, PEG-5 oleate, PEG-6 oleate, PEG-7 oleate, PEG-6 laurate, PEG-7 laurate, PEG-6 stearate, PEG-8 laurate, PEG-8 oleate, PEG-8 stearate, PEG-9 oleate, PEG-9 stearate, PEG-10 laurate, PEG-10 oleate, PEG-10 stearate, PEG-12 laurate, PEG-12 oleate, PEG-12 ricinoleate, PEG-12 stearate, PEG-15 stearate, PEG-15 oleate, PEG-20 laurate, PEG-20 oleate, PEG-20 stearate, PEG-25 stearate, PEG-32 laurate, PEG-32 oleate, PEG-32 stearate, PEG-30 stearate, PEG 4-100 monolaurate, PEG 4-100 monooleate, and PEG 4-100 monostearate.

PEG-Fatty Acid Diester Surfactants:

Polyethylene glycol (PEG) fatty acid diesters are also suitable for use as surfactants in the compositions of the present disclosure. Particularly preferred are PEG-fatty acid diesters with aliphatic C6-C22 carboxylic acids, whereby the polyethylene glycol comprises 6 to 60 ethylene oxide units per molecule. Representative PEG-fatty add diesters commercially available are: PEG-4 dilaurate, PEG-4 dioleate, PEG-6 dilaurate, PEG-6 dioleate, PEG-6 distearate, PEG-8 dilaurate, PEG-8 dioleate, PEG-8 distearate, PEG-10 dipalmitate, PEG-12 dilaurate, PEG-12 distearate, PEG-12 dioleate, PEG-20 dilaurate, PEG-20 dioleate, PEG-20 distearate, PEG-32 dilaurate, PEG-32 dioleate, and PEG-32 distearate.

PEG-Fatty Acid Mono- and Di-Ester Mixtures:

In general, mixtures of surfactants are also useful in the present disclosure, including mixtures of two or more commercial surfactant products. Particularly preferred are mixtures of PEG-fatty acid mono- and diesters with aliphatic C6-C22 carboxylic acids, whereby the polyethylene glycol comprises 6 to 60 ethylene oxide units per molecule. Several PEG-fatty acid esters are marketed commercially as mixtures or mono- and diesters. Representative surfactant mixtures commercially available are: PEG 4-150 mono, dilaurate; PEG 4-150 mono, dioleate; and PEG 4-150 mono, distearate.

Polyethylene Glycol (PEG) Glycerol Fatty Acid Esters:

In addition, PEG glycerol fatty acid esters are suitable surfactants in the context of the present disclosure, such as PEG-20 glyceryllaurate, PEG-30 glyceryllaurate, PEG-15 glyceryllaurate, PEG-40 glyceryllaurate. PEG-20 glyceryl stearate, PEG-20 glyceryl oleate, and PEG-30 glyceryl oleate. Particularly preferred are PEG glycerol fatty acid esters with aliphatic C8-C22 carboxylic acids, whereby the polyethylene glycol comprises 6 to 60 ethylene oxide units per molecule.

Polyethylene Glycol (PEG) Alkyl Ethers (Mono- and/or Diethers of Polyethylene Glycol):

Ethers of polyethylene glycol and alkyl alcohols are suitable surfactants for use in the present disclosure. Particularly preferred are PEG-fatty acid mono- and/or diethers with aliphatic C-12-C18 alcohols, whereby the polyethylene glycol comprises 6 to 60 ethylene oxide units per molecule. Some commercially available examples of these surfactants are: PEG-2 oleyl ether (oleth-2). PEG-3 oleyl ether (oleth-3), PEG-5 oleyl ether (oleth-5), PEG-10 oleyl ether (oleth-1 0), PEG-20 oleyl ether (oleth-20), PEG-4 lauryl ether (laureth-4); PEG-9 lauryl ether, PEG-23 lauryl ether (laureth-23), PEG-2 cetyl ether, PEG-10 cetyl ether, PEG-20 cetyl ether, PEG-2 stearyl ether, PEG-10 stearyl ether, and PEG-20 stearyl ether.

Polyethylene Glycol Sterol Ethers:

PEG-derivatives of sterols are suitable surfactants for use in the present disclosure. Examples of surfactants of this class are: PEG-24 cholesterol ether, PEG-30 cholestanol, PEG-25 phytosterol, PEG-5 soya sterol, PEG-10 soya sterol, PEG-20 soya sterol, and PEG-30 soya sterol.

Polyethylene Glycol Sorbitan Fatty Acid Esters:

A variety of PEG-sorbitan fatty acid esters are available and are suitable for use as surfactants in the present disclosure. Examples of these surfactants are: PEG-10 sorbitan laurate, PEG-20 sorbitan monolaurate, PEG-4 sorbitan monolaurate, PEG-80 sorbitan monolaurate, PEG-6 sorbitan monolaurate, PEG-20 sorbitan monopalmitate, PEG-20 sorbitan monostearate, PEG-4 sorbitan monostearate, PEG-8 sorbitan monostearate, PEG-6 sorbitan monostearate, PEG-20 sorbitan tristearate, PEG-60 sorbitan tetrastearate, PEG-5 sorbitan monooleate, PEG-6 sorbitan monooleate, PEG-20 sorbitan monooleate, PEG-40 sorbitan oleate, PEG-20 sorbitan trioleate, PEG-6 sorbitan tetraoleate, PEG-30 sorbitan tetraoleate, PEG-40 sorbitan tetraoleate, PEG-20 sorbitan monoisostearate, and PEG sorbitol hexaoleate.

Sugar Esters:

Esters of sugars, in particular mono-esters are suitable surfactants for use in the present disclosure. Examples of such surfactants are: Sucrose distearate/monostearate, Sucrose dipalmitate, Sucrose monostearate, Sucrose monopalmitate, Sucrose monolaurate, and Saccharose monolaurate.

Polyoxyethylene-Polyoxypropylene Block Copolymers:

The POE-POP block copolymers are a unique class of polymeric surfactants. The unique structure of the surfactants, with hydrophilic POE and lipophilic POP moieties in well-defined ratios and positions, provides a wide variety of surfactants suitable for use in the present disclosure. The generic term for these polymers is “poloxamer” (CAS 9003-11-6). These polymers have the formula: HO(C2H4O)a(C3H6O)b(C2H4O)aH, where “a” and “b” denote the number of polyoxyethylene and polyoxypropylene units, respectively.

Furthermore, amphoteric compounds such as fatty acid-amidoalkyl betaines with C2-C22 fatty acids are suitable surfactants.

The surfactant can also be, or include as a component, an ionic surfactant, including cationic, anionic and zwitterionic surfactants. Preferred anionic surfactants include fatty acid salts and bile salts. Preferred cationic surfactants include camitines. Specifically, preferred ionic surfactants include sodium oleate, sodium lauryl sulfate, sodium lauryl sarcosinate, sodium dioctyl sulfosuccinate, sodium cholate, sodium taurocholate; lauroyl carnitine; palmitoyl camitine; myristoyl camitine, alginate salts; propylene glycol alginate; lecithins and hydrogenated lecithins; lysolecithin and hydrogenated lysolecithins; lysophospholipids and derivatives thereof; phospholipids and derivatives thereon, salts of alkylsulfates; sodium docusate; carnitines; and mixtures thereof.

More specifically, preferred ionic surfactants are lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidylinositol, lysophosphatidic acid, lysophosphatidylserine, PEG phosphatidylethanolamine, PVP-phosphatidylethanolamine, stearoyl-2-actylate, stearoyllactylate, cholate, taurocholate, glycocholate, deoxycholate, taurodeoxycholate, chenodeoxycholate, glycodeoxycholate, glycochenodeoxycholate, taurochenodeoxycholate, ursodeoxycholate, tauroursodeoxycholate, glycoursodeoxycholate, cholylsarcosine, N-methyl taurocholate, caproate, caprylate, caprate, laurate, myristate, palmitate, oleate, ricinoleate, linoleate, linolenate, stearate, lauryl sulfate, teracecyl sulfate, docusate, lauroyl camitines, palmitoyl camitines, myristoyl camitines, and salts and mixtures thereof.

A co-surfactant, sometimes also referred to as a “co-emulsifier”, in the context of the present disclosure is equally a chemical compound which has both hydrophobic (lipophilic) and hydrophilic portions, but with the hydrophobic (lipophilic) nature predominating. It is intended to make the aqueous and oily phases in a microemulsion mutually soluble. Suitable co-surfactants in the context of the present disclosure have an HLB value below 10, preferably below 8 and even more preferred below 6. Co-surfactants can be any partial esters and/or partial ethers of polyhydric (polyvalent) alcohols, such as glycerol, propylenglycol (1,2-propanediol; 1,2-dihydroxypropane), ethyl-diglycol or even polyglycerols (such as digylcerol, triglyceryl, tetraglycerol etc.) with aliphatic carboxylic acids (fatty acids) or aliphatic alcohols (fatty alcohols).

Further co-surfactants, which can be grouped into some general chemical classes, are given below. It should be emphasized that the present disclosure is not limited to the co-surfactants indicated herein, which show representative, but not exclusive, lists of available co-surfactants.

Mono-Glycerides:

A particularly important class of co-surfactants is the class of mono-glycerides, which are generally lipophilic. Particulary preferred are mixtures of monoglycerides with aliphatic CS-C22 carboxylic acids. Examples of this class of co-surfactants are: Monopalmitolein (C16:1), Monoelaidin (C18:1), Monocaproin (C6), Monocaprylin, 10 Monocaprin, Monolaurin, Glyceryl monomyristate (C14), Glyceryl monooleate (C18:1), Glyceryl monooleate, Glyceryl monolinoleate, Glyceryl ricinoleate, Glyceryl monolaurate, Glyceryl monopalmitate, Glyceryl monostearate, Glyceryl monopalmate, Glycerol monostearate, Glyceryl caprylate, and Glyceryl caprate as well as mixtures thereof.

Polyglycerized Fatty Acids:

Polyglycerol esters of fatty acids, in particular Polyglycerol mono-esters, are also suitable co-surfactants for the present disclosure. Particulary preferred are mixtures of polyglycerol esters with aliphatic C8-C22 carboxylic acids. Examples of suitable polyglyceryl esters commercially available are: Polyglyceryl-2 stearate, Polyglyceryl-2 oleate, Polyglyceryl-2 isostearate, Polyglyceryl-3 oleate, Polyglyceryl-4 oleate, Polyglyceryl-4 stearate, Polyglyceryl-6 oleate, Polyglyceryl-2 dioleate, and Polyglyceryl-6 dioleate.

Propylene Glycol Fatty Acid Esters:

Partial esters of propylene glycol and fatty acids, in particular mono-esters, are suitable co-surfactants for use in the present disclosure. Particularly preferred are mixtures of propylene glycol esters with aliphatic C8-C22 carboxylic acids. Examples of co-surfactants of this class are: Propylene glycol monocaprylate, Propylene glycol monolaurate, Propylene glycol oleate, Propylene glycol myristate, Propylene glycol monostearate, Propylene glycol hydroxy stearate, Propylene glycol ricinoleate, Propylene glycol isostearate, Propylene glycol monooleate, Propylene glycol dicaprylate/dicaprate, Propylene glycol dioctanoate, Propylene glycol caprylate/caprate, Propylene glycol dilaurate, Propylene glycol distearate, Propylene glycol dicaprylate, and Propylene glycol dicaprate.

A lipophilic phase in the context of the present disclosure is understood to mean a water-immiscible liquid. The lipophilic phase may also be referred to as being a lipidic phase. For oral pharmaceutical dosage forms of the present disclosure includes a lipophilic component, the lipophilic component is preferably a triglyceride or a mixture of a triglyceride and a diglyceride. Suitable lipophilic phases are preferably di- and triacylglycerides of aliphatic carboxylic acids (fatty acids) with 4 to 22 carbon atoms, in particular with 6 to 22 carbon atoms, and also mixtures thereof.

Preferred di-glycerides in the context of the present disclosure are mixtures of di-glycerides with aliphatic C8-C22 carboxylic acids. Examples are: Glyceryl dioleate, Glyceryl dipalmitate, Glyceryl dilaurate, Glyceryl dilinoieate, Glyceryl dicaprylate, Glyceryl dicaprate, Glyceryl caprylate/caprate, Glyceryl distearate, Glyceryl stearate/palmitate, Glyceryl oleate/linoleate and Glyceryl dimyristate,

Preferred triglycerides are those which solidify at ambient room temperature, with or without addition of appropriate additives, orthose which in combination with particular surfactants and/or co-surfactants and/or active ingredients solidify at room temperature. Examples of triglycerides suitable for use in the present disclosure are: Aceituno oil, Almond oil, Araehis oil, Babassu oil, Beeswax, Blackcurrant seed oil, Borage oil, Buffafo ground oil, Candlenut oil, Canola oil, Castor oil, Chinese vegetable tallow oil, Cocoa butter, Coconut oil, Coffee seed oil, Corn oil, Cottonseed Oil, Crambe oil, Cup-hea species oil, Evening primrose oil, Grapeseed oil, Groundnut oil, Hemp seed oil, Illipe butter, Kapok seed oil, Linseed oil, Menhaden oil, Mowrah butter, Mustard seed oil, Oiticica oil, Olive oil, Palm oil, Palm kernel oil, Peanut oil, Poppy seed oil, Rapeseed oil, Rice bran oil, Safflower Oil, Sal fat, Sesame oil. Shark liver Oil, Shea nut oil, Soybean Oil, Stillingia oil, Sunflower oil, Tall oil, Tea seed oil, Tobacco seed oil, Tung oil (China wood oil), Ucuhuba, Vemonia oil, Wheat germ Oil, Hydrogenated castor oil, Hydrogenated coconut oil, Hydrogenated cottonseed oil, Hydrogenated palm oil, Hydrogenated soybean oil, Hydrogenated vegetable oil, Hydrogenated cottonseed and castor oil, Partially hydrogenated soybean oil, Partially hydrogenated soy and cottonseed oil, Glyceryl mono-, di-, tri-behenate, Glycerol tributyrate, Glyceryl tricaproate, Glyceryl tricaprylate, Glyceryl tricaprate, Glyceryl triundecanoate, Glyceryl trilaurate, Glyceryl trimyistate, Glyceryl tripalmitate, Glyceryl tristearate, Glyceryl triarchidate, Glyceryl trimyristoleate, Glyceryl tripalmitoleate, Glyceryl trioleate, Glyceryl trilinoleate, Glyceryl trilinolenate, Glyceryl tricaprylate/caprate, Glyceryl tricaprylate/caprate/laurate, Glyceryl tricaprylate/caprate/linoleate, Glyceryl tricaprylate/caprate/stearate, Glyceryl tricaprylate/laurate/stearate, Glyceryl 1,2-caprylate-3-linoleate, Glyceryl 1,2-caprate-3-stearate, Glyceryl 1,2-laurate-3-myristate, Glyceryl 1, 2-myristate-3-laurate, Glyceryl1, 3-palmitate-2-butyrate, Glyceryl1,3-stearate-2-caprate, Glyceryl 1, 2-linoleate-3-caprylate.

Fractionated triglycerides, modified triglycerides, synthetic triglycerides, and mixtures of triglycerides are also within the scope of the present disclosure. Preferred triglycerides include vegetable oils, fish oil, animal fats, hydrogenated vegetable oils, partially hydrogenated vegetable oils, medium and long-chain triglycerides, and structured triglycerides.

Furthermore, the following compounds may be suitable as lipophilic phase: low-viscosity and high-viscosity aliphatic hydrocarbons, and also in particular oleic acid oleyl ester, isooctyl stearate, lauric acid hexyl ester, di-n-butyl adipate, isopropyl myristate, isopropyl palmitate and isopropyl stearate, oleyl alcohol, ethereal oils, isopropyl caprylate, isopropyl caprinate and isopropyllaurate.

### Complete Systems Composed of Surfactant, Co-Surfactant and Lipophilic Phase

Several commercial surfactant and/or co-surfactant compositions contain small to moderate amounts of di- and triglycerides, typically as a result of incomplete reaction of a triglyceride starting material in, for example, a transesterification reaction. Such commercial surfactant and/or co-surfactant compositions, while nominally referred to as “surfactants” and/or “co-surfactant”, may be suitable to provide in addition to the surfactant and/or co-surfactant part of the system—all or part of the lipophilic component, i.e. the di- and triglyceride component, for the compositions of the present disclosure.

Still other commercial surfactant and/or co-surfactant compositions having significant di- and triglyceride content are known to those skilled in the art. It should be appreciated that such compositions, which contain di- and triglycerides as well as surfactants and/or co-surfactants, may be suitable to comprise the surfactant, co-surfactant and lipophilic phase of the compositions of the present disclosure. Typical examples are so-called macrogolglycerides (or polyoxyethylated glycerides) with different kinds of fatty acids. Macrogolglycerides are mixtures of mono-esters, di-esters and tri-esters of glycerol and mono-ester and diester of PEG (=Polyethylene glycol, Macrogol, Polyoxyethlene, Poly Ethylene Oxide, Polyglycol) with fatty acids, whereby the molecular mass of the PEG can be defined as well as the nature of the fatty acids. Macrogolglycerides can be obtained by a partial hydrolysis/esterification reaction of triglycerides using the respective macrogol. Alternatively, macragolglycerides can be obtained by esterification of glycerol and the macrogol and the corresponding free fatty acids. As triglycerides a variety of natural and/or hydrogenated oils can be used. Most commonly, the oils used are castor oil or hydrogenated castor oil or an edible vegetable oil such as corn oil, olive oil, peanut oil, palm kernel oil, apricot kernel oil, or almond oil, or the corresponding hydrogenated vegetable oil.

Typically, such transesterification products of oils and polyethylenglycol (or other polyalcohols) are named by their educts: PEG-20 castor oil, PEG-23 castor oil, PEG-30 castor oil, PEG-35 castor oil, PEG-38 castor oil. PEG-40 castor oil, PEG-50 castor oil, PEG-56 castor oil, PEG-7 hydrogenated castor oil, PEG-1 0 hydrogenated castor oil. PEG-20 hydrogenated castor oil, PEG-25 hydrogenated castor oil, PEG-30 hydrogenated castor oil, PEG-40 hydrogenated castor oil, PEG-45 hydrogenated castor oil, PEG-50 hydrogenated castor oil, PEG-60 hydrogenated castor oil, PEG-80 hydrogenated castor oil, PEG-8 corn oil, PEG-20 corn oil, PEG-20 almond oil, PEG-25 trioleate, PEG-40 palm kernel oil, PEG-60 corn oil, PEG-60 almond oil, PEG-8 caprylic/capric glycerides, Lauroyl macrogol-32 glyceride (=PEG-32 hydrogenated palm kernel oil, e.g. Gelucire® 44/14), Stearoyl macrogol glyceride (e.g. Gelucire® 50/13)

Examples of commercial co-surfactant compositions of mono-glycerides, in addition containing di- and triglycerides include some members of the co-surfactant families Maisines® (Gattefosse) and Imwitors® (Huls). These commercial compositions may be used for providing the co-surfactant and the lipophilic phase in one composition. Specific examples of these compositions are: Maisine® 35-1 (linoleic glycerides) and Imwitor® 742 (caprylic/capric glycerides).

Aliphatic Carboxylic Acids with 6 to 22 Carbon Atoms:

In the context of the present disclosure, aliphatic carboxylic acids with 6 to 22 carbon atoms are understood as aliphatic C6-C22 carboxylic acids. Thus preferably carboxylic acids selected from the group containing caproic acid (C6), caprylic acid (C8), capric acid (C10), lauric acid (C12), mystic acid (C14), palmitic acid (C16), stearic acid (C18), arachidic acid (C20), and behenic acid (C22), as well as the corresponding unsaturated carboxylic acids, such as palmitoleic acid (C16), oleic acid (C18), linoleic acid (C18), linolenic acid (C18), eicosenoic acid (C20), individually or as a mixture, are used. Particularly preferably, the saturated carboxylic acids are selected.

Aliphatic Alcohols with 12 to 18 Carbon Atoms:

In the context of the present disclosure, aliphatic alcohols with 12 to 18 carbon atoms are understood as aliphatic C12-C18 alcohols. Thus preferably alcohols selected from the group containing lauryl alcohol (C12), myristyl alcohol (C14), cetyl alcohol (C16), stearyl alcohol (C18), oleyl alcohol (C18), linoleyl alcohol (C18) and linolenyl alcohol (C18), individually or as a mixture, are used. Particularly preferably, the saturated alcohols are selected.

Aliphatic alcohols with 12 to 22 carbon atoms: In the context of the present disclosure, aliphatic alcohols with 12 to 22 carbon atoms are understood as aliphatic C12-C22 alcohols. Thus preferably alcohols selected from the group containing lauryl alcohol (C12), myristyl alcohol (C14), cetyl alcohol (C16), stearyl alcohol (C18), arachidyl alcohol (C20), behenyl alcohol (C22), oleyl alcohol (C18), linoleyl alcohol (C18) and linolenyl alcohol (C18), individually or as a mixture, are used. Particularly preferably, the saturated alcohols are selected.

The hydrophilic phase in the context of the present disclosure is understood in particular to mean an aqueous phase which is preferably supplied by the physiological liquid of the digestion medium and/or by an aqueous liquid ingested in parallel with the food and/or the pharmaceutical preparation.

Enzymes or enzyme mixtures with at least lipolytic activity in the context of the present disclosure are understood to mean physiologically acceptable enzyme mixtures which contain at least one lipase. Furthermore, the enzymes or enzyme mixtures may however also have proteolytic activity in addition to the lipolytic activity, i.e. contain at least one protease, and/or amylolytic activity, i.e. contain at least one amylase. Enzymes or enzyme mixtures may be use which exhibit (i) purely lipolytic; or (ii) lipolytic and proteolytic; or (iii) lipolytic and amylolytic; or (iv) lipolytic, proteolytic and amylolytic activity. Suitable enzymes or enzyme mixtures may be of any animal or microbiological origin. Particularly suitable enzymes are from porcine origin. The enzyme mixtures with at least lipolytic, and optionally also proteolytic and/or amylolytic activity used in the context of the present disclosure may therefore be of purely microbial origin or of purely animal origin, or alternatively represent a mixture of enzymes of animal and microbial origin.

In the case of lipase-containing enzyme products of non-animal origin as well as preparations thereof, these are enzyme mixtures comprising at least one lipase and optionally also at least one protease and/or amylase. These enzymes may be plant-derived or of fungal or bacterial origin. These lipases, proteases and/or amylases may for example be obtained by fermentation of optionally recombinant bacteria or fungi. The lipase-containing enzyme products may be composed of purely microbial derived enzyme preparations (i.e. enzymes obtained from fungi or bacteria) or enzyme preparations obtained from plants, but also of synthetic mixtures of enzyme preparations from plants, bacteria and/or fungi, optionally produced recombinantly in a microbial system. Furthermore, the recombinantly produced enzyme may be an enzyme variant or a mutated enzyme being functionally equivalent or having structural features similar to a naturally occurring enzyme.

By “recombinantly produced microbial enzyme”, in particular “recombinantly produced lipase, amylase or protease”, is meant an enzyme produced by way of recombinant DNA-technology, the enzyme being of microbial origin, i.e. obtained from fungi or bacteria. In the context of this disclosure suitable lipases are recombinantly produced microbial lipases that possess lipolytic activity, preferably at relatively low pH. In the context of this disclosure suitable proteases are recombinantly produced microbial proteases that possess proteolytic activity, preferably at relatively low pH. In the context of this disclosure suitable amylases are recombinantly produced microbial amylases that possess amylolytic activity, preferably at relatively low pH.

The recombinantly produced microbial enzyme, i.e. the lipase, amylase or protease, may be an enzyme variant or a mutated enzyme being functionally equivalent or having structural features similar to a naturally occurring enzyme.

Preferred recombinantly produced microbial lipases are lipases derived from fungi, e.g. from Humicola, Rhizomucor, Rhizopus, Geotrichum or Candida species, in particular Humicola lanuginosa (Thermomyces lanuginosa), Rhizomucor miehei, Rhizopus javanicus, Rhizopus arrhizus, Rhizopus oryzae, Rhizopus delamar, Candida cylindracea, Candida rugosa or Geotrichum candidum; or may be derived from bacteria, e.g. from Pseudomonas, Burkholderia or Bacillus species, in particular Burkholderia cepacia. Most preferred are lipases derived from a strain of Humicola lanuginosa (Thermomyces lanuginosa) or Rhizomucor miehei.

Lipases of microbial origin which can be used in the context of the present disclosure and their production by e.g. recombinant technology are described in e.g. EP Publication Nos. 0600868, 0238023, 0305216, 0828509, 0550450, 1261368, 0973878 and 0592478, which publications are hereby included by reference.

Preferred recombinantly produced microbial amylases are amylases derived from fungi, e.g. from Aspergillus or Rhizopus species, in particular Aspergillus niger or Aspergillus oryzae; or may be derived from bacteria, e.g. from Bacillus species, in particular Bacillus subtilis. Most preferred are amylases derived from a strain of Aspergillus oryzae.

Amylases of microbial origin which can be used in the context of the present disclosure and their production by recombinant technology are described in e.g. EP Publication No. 0828509, which publication is hereby included by reference.

Preferred recombinantly produced microbial proteases are proteases derived from fungi, e.g. from Aspergillus or Rhizopus species, In particular Aspergillus melleus, Aspergillus oryzae, Aspergillus niger, or Rhizopus oryzae; or may be derived from bacteria, e.g. from Bacillus species, in particular Bacillus subtilis. Most preferred are proteases derived from a strain of Aspergillus melleus.

Proteases of microbial origin which can be used in the context of the present disclosure are described in e.g. Publication EP 1186658 and Pariza & Johnson, “Evaluating the safety of microbial enzyme preparations used in food processing: update for a new century.” Regul Toxicol Pharmacol. 2001 April; 33(2): 173-86, which are hereby included by reference.

The recombinantly produced microbial enzyme, i.e. lipase, amylase or protease, may be obtained by fermentation of a fungal cell, e.g. belonging to the genus Aspergillus, such as A. niger, A. oryzae, or A. nidulans; a yeast cell, e.g. belonging to a strain of Saccharomyces, such as S. cerevisiae, or a methylotrophic yeast from the genera Hansenula, such as H. polymorpha, or Phichia, such as P. pastoris; or a bacterial cell, e.g. belonging to a strain of Bacillus, such as B. subtilis, or B. lentus; the cell being transformed with the gene encoding the microbial lipase. Most preferred host organisms are members of Aspergillus oryzae.

An enzyme variant or mutated enzyme is obtainable by alteration of the DNA sequence of the parent gene or its derivatives. The enzyme variant or mutated enzyme may be expressed and produced when the DNA nucleotide sequence encoding the respective enzyme is inserted into a suitable vector in a suitable host organism. The host organism does not necessarily have to be identical to the organism from which the parent gene originated. The methods for introducing mutations into genes are well known in the art, vide e.g. Patent Application EP 0407225.

Preferred lipase variants or mutated lipases are obtainable from parent microbial lipases. In a preferred embodiment the parent lipase is derived from a fungus, e.g. a strain of Humicola or Rhizomucor, preferably a strain of Humicola lanuginosa or a strain of Rhizomucor miehei. In another preferred embodiment the parent lipase is derived from yeast, e.g. derived from a strain of Candida. In a further preferred embodiment the parent lipase is derived from a bacterium, e.g. derived from a strain of Pseudomonas. More preferred lipase variants or mutated lipases are lipase variants of parent lipases comprising a trypsin-like catalytic triad including an active serine residue located in a predominantly hydrophobic, elongated binding pocket of the lipase molecule, wherein the electrostatic charge and/or hydrophobicity of a lipid contact zone comprising residues located in the vicinity of the lipase structure containing the active serine residue, which residues may participate in the interaction with the substrate at or during hydrolysis, has been changed by deleting or substituting one or more negatively charged amine acid residues by neutral or positively charged amino acid residue(s), and/or by substituting one or more neutral amino acid residues by positively charged amino acid residue(s), and/or by deleting or substituting one or more hydrophobic amino acid residues by hydrophobic amino acid residue(s).

Pharmaceutically compatible auxiliaries, carriers and/or excipients in the context of the present disclosure are preferably selected from the group consisting of free polyethylene glycols having an average molecular weight of about 200 to about 6000, glycerol, lower alcohols, in particular straight-chain or branched C1-C4-alcohols such as 2-propanol, sugars, such as lactose, sucrose or dextrose; polysaccharides, such as maltodextrin or dextrates; starches; cellulosics, such as microcrystalline cellulose or microcrystalline cellulose/sodium carboxymethyl cellulose; inorganics, such as dicalcium phosphate, hydroxyapatite, tricalcium phosphate, talc, or titania; and polyols, such as mannitol, xylitol, sorbitol or cyclodextrin; and mixtures of the aforementioned substances.

The present disclosure discloses pharmaceutical compositions for oral administration, which are self-emulsifiable on contact with a hydrophilic phase and a lipophilic phase, said composition comprising:
(i) enzymes or enzyme mixtures with at least lipolytic activity,
(ii) at least one surfactant,
(iii) at least one co-surfactant.
(iv) optionally a lipophilic phase, and
(v) optionally an antioxidant.

Preferably the pharmaceutical composition of the present disclosure comprises enzymes or enzyme mixtures with at least lipolytic activity, a surfactant at least one agent having an HLB value above 6 and below 18, a co-surfactant at least one agent having an HLB-value below 10, and a lipophilic phase, wherein the surfactant, co-surfactant and lipophilic phase in combination has an HLB value of about 4 to 16, and a melting point greater than or equal to 20° C., preferably greater than or equal to 25° C.

The surfactant is preferably chosen from the group consisting of polyethylene glycol fatty acid esters; polyethylene glycol glycerol fatty acid esters; polyethylene glycol alkyl ethers, polyethylene glycol sterol ethers, polyethylene glycol sorbitan fatty acid esters, sugar esters, polyoxyethylene-polyoxypropylene block copolymers, ionic surfactants and mixtures thereof. Even more preferred, the surfactant is chosen from the group consisting of polyethylene glycol (PEG) fatty acid mono- and/or di-esters with aliphatic C6-C22 carboxylic acids; polyethylene glycol (PEG) glycerol fatty acid esters with aliphatic C8-C22 carboxylic acids; polyethylene glycol (PEG) alkyl mono- and/or di-ethers with aliphatic C12-C18 alcohols, and mixtures thereof. In particular, the surfactant used is represented by a mixture of polyethylene glycol (PEG) mono- and di-esters with aliphatic C6-C22 carboxylic acids and/or polyethylene glycol (PEG) monoand di-ethers with aliphatic C12-C18 alcohols, whereby the polyethylene glycol (PEG) comprises 6 to 60 ethylene oxide units per molecule (PEG-6 to PEG-60, also named as PEG 300 to PEG 3000), preferably by a mixture of polyethylene glycol mono- and di-esters with aliphatic C6-C22 carboxylic acids, whereby the polyethylene glycol comprises 6 to 40 ethylene oxide units per molecule.

The co-surfactant of the system is preferably chosen from the group consisting of mono-acylglycerides, monoethers of glycerol, partial esters of propylenglycol, partial esters of polyglycerol, partial esters of ethyl diglycol and mixtures thereof. Even more preferred, the co-surfactant chosen from the group consisting of mono-acylglycerides with aliphatic C6-C22 carboxylic acids, mono-ethers of glycerol ethers with aliphatic C12-C18 alcohols, partial esters of propylenglycol with aliphatic C6-C22 carboxylic acids, partial esters of polyglycerol with aliphatic C6-C22 carboxylic acids, and mixtures thereof. Particularly preferred co-surfactants are monoacylglycerides of aliphatic C6-C22 carboxylic acids and/or monoethers of glycerol with aliphatic C12-C22 alcohols, especially monoacylglycerides of aliphatic C6-C22 carboxylic adds.

The lipophilic phase is preferably represented by di- and/or triacylglycerides, preferably di- and/or triacylglycerides with aliphatic C6-C22 carboxylic acids.

The pharmaceutical composition according to the present disclosure comprises

2 to 90% by weight surfactants as defined above,

5 to 60% by weight co-surfactants as defined above, and

0 to 70% by weight of the lipophilic phase as defined above,

wherein the components surfactant, co-surfactant and the lipophilic phase together make up 10% to 95% by weight of the pharmaceutical composition, alternatively 20-60%, alternatively 25-45%, alternatively 30-45%, alternatively 31-41%, alternatively 31-39%, alternatively about 36%, or alternatively 36%.

The pharmaceutical composition or oral pharmaceutical dosage forms of the present disclosure comprises an lipase-containing enzyme at least 55% by weight of the total composition, alternatively 55-75%, alternatively 59-69%, alternatively 61-69%, alternatively 63-67%, alternatively about 64%, or alternatively 64%.

In a preferred embodiment, the pharmaceutical composition according to the invention comprises a macrogolglycerides mixture representing the surfactant, co-surfactant and lipophilic phase, whereby the macrogolglycerides are a mixture of mono-, di- and tri-acylglycerides and polyethylene glycol (PEG) mono- and diesters of aliphatic C6-C22 carboxylic acids, and also possibly small proportions of glycerol and free polyethylene glycol. A particularly suitable macrogolglycerides is Gelucire® 44/14.

The polyethylene glycol (PEG) contained in the macrogolglyceride mixture is preferably PEG which has on average 6 to at most 40 ethylene oxide units per molecule or a molecular weight of between 200 and 2000.

One further aspect of the present disclosure provides for a pharmaceutical composition comprising surfactant, co-surfactant and lipophilic phase, wherein the combination of the surfactant, co-surfactant and lipophilic phase has an HLB value greater than or equal to 10 and a melting point greater than or equal to 30° C. In a preferred embodiment, the combination of the surfactant, co-surfactant and lipophilic phase has an HLB value of 10 to 16, preferably of 12 to 15, and has a melting point of between 30 and 60° C., preferably between 40 and 50° C.

In particular, the combination of the surfactant, co-surfactant and lipophilic phase is characterized by HLB value and melting point is a mixture of mono-, di- and triacylglycerides and mono- and diesters of polyethylene glycol (PEG) with aliphatic carboxylic acids with 8 to 20 carbon atoms, whereby the polyethylene glycol preferably has about 6 to about 32 ethylene oxide units per molecule, and the system optionally contains free glycerin and/or free polyethylene glycol. The HLB value of such a combination is preferably regulated by the chain length of the PEG. The melting point of such a combination is regulated by the chain length of the fatty acids, the chain length of the PEG and the degree of saturation of the fatty-acid chains, and hence the starting oil for the preparation of the macrogolglyceride mixture.

“Aliphatic C8-C18 carboxylic acids” designates mixtures in which caprylic acid (C8), capric acid (C10), lauric acid (C12), myristic acid (C14), palmitic acid (C16) and stearic acid (C18) are contained in a significant and variable proportion, if these acids are saturated, and the corresponding unsaturated C8-C18 carboxylic acids. The proportions of these fatty acids may vary according to the starting oils.

Such a mixture of mono-, di- and triacylglycerides and mono- and diesters of polyethylene glycol (PEG) with aliphatic carboxylic acids with 8 to 18 carbon atoms can for example be obtained by a reaction between a polyethylene glycol with a molecular weight of between 200 and 1500 and a starting oil, the starting oil consisting of a triglyceride mixture with fatty acids which are selected from the group containing caprylic acid, capric acid, lauric acid, myristic acid, palmitic acid, stearic acid, oleic acid and linolenic acid, individually or as a mixture. Optionally, the product of such a reaction may also contain small proportions of glycerin and free polyethylene glycol.

Such a mixture is commercially available for example under the trade name Gelucire®. One advantageous embodiment of the invention provides that, of the products known under the trade name Gelucire®, in particular “Gelucire® 50/13” and/or “Gelucire® 44/14” represent suitable mixtures for use in the pharmaceutical preparations according to the present disclosure.

Gelucire® 50/13 is a mixture with mono-, di- and triacylglycerides and mono- and diesters of polyethylene glycol, with palmitic acid (C16) and stearic acid (C18) at 40% to 50% and 48% to 58%, respectively making up the major proportion of bound fatty acids. The proportion of caprylic acid (C8) and capric acid (C10) is less than 3% in each case, and the proportion of lauric add (C12) and myristic acid (C14), in each case is less than 5%.

A preferred embodiment of the present disclosure provides for a pharmaceutical composition comprises a mixture of mono-, di- and triacylglycerides and polyethylene glycol mono- and diesters of aliphatic C8-C18 carboxylic acids and also possibly small proportions of glycerin and free polyethylene glycol, the system having a melting point between 46° C. and 51° C. and an HLB value of around 13.

Gelucire® 44/14 is a mixture with mono-, di- and triacylglycerides and mono- and diesters of polyethylene glycol, the respective proportions of palmitic acid (C16) being 4 to 25%, stearic acid (C18) 5 to 35%, caprylic add (C8) less than 15%, capric acid (C10) less than 12%, lauric acid (C12) 30 to 50% and myristic acid (C14) 5 to 25%. Gelucire® 44/14 can for example be prepared by an alcoholysis/esterification reaction using palm kernel oil and polyethylene glycol 1500.

One preferred embodiment of the present disclosure provides for a pharmaceutical composition comprising a mixture of mono-, di- and triacylglycerides and polyethylene glycol mono- and diesters of aliphatic C8-C18 carboxylic acids and also possibly small proportions of glycerin and free polyethylene glycol, the system having a melting point between 42° C. and 48° C. and an HLB value of around 14.

In an alternative embodiment, the pharmaceutical composition of the present disclosure is characterized in that an ionic surfactant is used as surfactant. Preferably, the ionic surfactant is selected from the group consisting of lecithin, lysolecithin, phosphatidylcholine, phosphatidylethanolamine, phosphatidylglycerol, phosphatidylserine, lysophosphatidylcholine, lysophosphatidylethanolamine, lysophosphatidylglycerol, lysophosphatidylinositol, lysophosphatidic acid, lysophosphatidylserine, and mixtures thereof.

For the present pharmaceutical compositions or oral pharmaceutical dosage forms according to the present disclosure, preferably solid orally administered dosage forms may be selected, for example powders, pellets, granules, tablets, or microspheres, which if desired may be filled into capsules or sachets or may be compressed to form tablets. Granules are preferably produced by melt granulation. Tablets are usually made from the powder or the melt granules. Pellets can be produced either by exploiting the thermoplastic properties of the auxiliaries in a heavy-duty mixer (melt pelletisation) or by traditional methods e.g. extrusion (e.g. melt extrusion or wet extrusion) and spheronisation. If individual enzyme types are present and are obtained separately, such as a lipase, a protease or an amylase from microbial origin, these may in this case be present together or spatially separated from each other. If the individual enzymes are not spatially separated from each other, dry processing and/or storage is preferred.

The pharmaceutical compositions or oral pharmaceutical dosage forms according to the present disclosure, which are self-emulsifiable on contact with a hydrophilic phase and optionally a lipophilic phase, contain enzymes or enzyme mixtures with at least lipolytic activity as active substance. In a preferred variant of the present disclosure, the enzymes or enzyme mixtures may however, in addition to the lipolytic activity, also have proteolytic activity, i.e. contain at least one protease, and/or amylolytic activity, i.e. contain at least one amylase.

In one preferred embodiment, the lipolytic activity of the enzymes or enzyme mixtures is provided by a microbial lipase. In other embodiments, the lipolytic activity of the enzymes or enzyme mixtures is provided by a non-human mammalian lipase. In another preferred embodiment, the lipolytic activity of the enzymes or enzyme mixtures is provided by a porcine lipase.

In another embodiment, the pharmaceutical composition or oral pharmaceutical dosage forms contains enzymes or enzyme mixtures which are pancreatin and/or pancreatin-like, preferably pancreatin-containing mixtures of digestive enzymes. Preferably, the pancreatin and/or pancreatin-like mixtures of digestive enzymes make up at least 55% by weight of the total composition, alternatively 55-75%, alternatively 59-69%, alternatively 61-69%, alternatively 63-67%, alternatively about 64%, or alternatively 64%.

Alternatively, the enzyme mixture used is a mixture of at least one microbial lipase and one or more microbial enzymes from the group of proteases and amylases is used as enzyme mixture. Alternatively, the enzyme mixture used is a mixture of at least one porcine lipase and one or more porcine enzymes from the group of proteases and amylases is used as enzyme mixture. Alternatively, the enzyme mixture used is a mixture of at least one non-human mammalian lipase and one or more non-human mammalian enzymes from the group of proteases and amylases is used as enzyme mixture.

In one embodiment, the enzyme mixture used is purely of microbial origin. Examples of such physiologically acceptable bacterial and/or fungal enzymes have already been described in the prior art, together with procedures how to obtain these enzymes and with their use for the treatment of maldigestion. For example such synthetic mixtures of lipase, protease and amylase, each of which are microbially obtained, and also pharmaceutical preparations containing these mixtures are described in international patent application WO 02/060474 and patent application EP 0 828 509.

In some embodiments, the pharmaceutical composition contains microbial enzymes making up. 5 80%, in particular 20-60% by weight, of the pharmaceutical composition.

In some embodiments, the enzyme mixture used is purely of porcine origin. Examples of such physiologically acceptable porcine enzymes and isolation thereof are known by those skilled in the art.

In the context of the present disclosure, most preferred are those mixtures of digestive enzymes with lipolytic, proteolytic and amylolytic activity, the properties of which are identical or close to those of pancreatin. Pancreatin-containing mixtures of digestive enzymes and also in particular pancreatin itself are therefore preferred in the context of the present disclosure as disclosed above.

However, it is possible to add to the pancreatin or the pancreatin-containing mixtures of digestive enzymes if desired one or more microbial enzymes, such as microbial-produced lipases, proteases and/or amylases obtained from microbial sources. Suitable microbial enzymes for the sole use as enzyme mixture or even as addition to pancreatin or the pancreatin-containing mixtures of digestive enzymes are in particular bacterial or fungal enzymes, such as from the species Bacillus or Pseudomonas, or from fungal cultures, such as from the species Aspergillus, Humicola or Rhizomucor. Preferably, the microbial enzymes, in particular the microbial lipase, are recombinantly produced. In a further preferred variant of the present disclosure the microbial lipase is a lipase variant or a mutated lipase.

The present disclosure furthermore relates to the use of surfactant combination comprising

at least one surfactant,

at least one co-surfactant, and

optionally a lipophilic phase

for stabilizing the lipolytic activity in the acidic pH range and/or for improving the lipolytic activity of solid pharmaceutical preparations containing enzymes or enzyme mixtures with at least lipolytic activity, preferably pancreatin or pancreatin-like mixtures of digestive enzymes.

The possible further configurations of the surfactant combination to be used. consisting of surfactant, co-surfactant and lipophilic phase, correspond to the embodiments already mentioned for the self-emulsifiable pharmaceutical preparation according to the present disclosure. These surfactant combinations are designed to the used in conjunction with lipolytic enzymes described herein.

In embodiments, the present pharmaceutical compositions can comprise an antioxidant. The term “antioxidants” is known per se to the person skilled in the art (see e.g. Römpp-Lexikon der Chemie [Lexicon of Chemistry], 9th edition, 1989, Georg-Thieme-Verlag, Stuttgart) and refers to substances which are intended to inhibit or prevent undesired changes brought about by oxygen or other oxidative processes. According to the present disclosure, suitable antioxidants for use in the present pharmaceutical compositions are sparingly water-soluble antioxidants, i.e. antioxidants whose solubility in water at 20° C. is not more than 1 g/l. Suitable antioxidants are primarily the lipophilic substances tocopherol, tocopherol acetate, tocotrienol, ascorbyl palmitate, ascorbyl stearate, t-butylhydroquinone, t-butylhydroxyanisole, t-butylhydroxytoluene, octyl gallate or dodecyl gallate or combinations thereof.

In one embodiment, the present pharmaceutical composition comprises: an enzyme or enzyme mixtures with at least lipolytic activity; a surfactant comprising a mixture of polyethylene glycol mono- and di-esters with aliphatic C6-C22 carboxylic acids, wherein the polyethylene glycol comprises 6 to 40 ethylene oxide units per molecule; a co-surfactant comprising monoacylglycerides of aliphatic C6-C22 carboxylic acids and/or monoethers of glycerol with aliphatic C12-C22 alcohols; and optionally, an antioxidant comprising tocopherol, tocopherol acetate, tocotrienol, or t-butylhydroxyanisole.

In another embodiment, the present pharmaceutical composition comprises: an enzyme or enzymes mixtures with at least lipolytic activity; a complete system composed of a surfactant, a co-surfactant, and lipophilic phase comprising one or more macrogolglycerides; and optionally, an antioxidant comprising tocopherol, tocopherol acetate, tocotrienol, or t-butylhydroxyanisole.

In a further embodiment, the present pharmaceutical composition comprises: an enzyme or enzyme mixtures with at least lipolytic activity; Stearoyl macrogol glyceride and/or Lauroyl macrogol-32 glyceride (PEG-32 hydrogenated palm kernel oil, e.g. Gelucire® 44/14); polyethylene glycol, particularly PEG 4000; and optionally, an antioxidant comprising tocopherol, tocopherol acetate, tocotrienol, or t-butylhydroxyanisole.

In a yet further embodiment, the present pharmaceutical composition comprises: an enzyme or enzyme mixtures with lipolytic, proteolytic, and amylolytic activity; a surfactant comprising a mixture of polyethylene glycol mono- and di-esters with aliphatic C6-C22 carboxylic acids, wherein the polyethylene glycol comprises 6 to 40 ethylene oxide units per molecule; a co-surfactant comprising monoacylglycerides of aliphatic C6-C22 carboxylic acids and/or monoethers of glycerol with aliphatic C12-C22 alcohols; and optionally, an antioxidant comprising tocopherol, tocopherol acetate, tocotrienol, or t-butylhydroxyanisole.

In embodiments of the present disclosure, the pharmaceutical composition comprises an enzyme or enzyme mixture with at least lipolytic activity, and optionally, proteolytic and/or amylolytic activity wherein the an enzyme or enzyme mixture comprises at least 55% by weight of the total composition, alternatively 55-75%, alternatively 59-69%, alternatively 61-69%, alternatively 63-67%, alternatively about 64%, or alternatively, 64%.

In embodiments of the present disclosure, the pharmaceutical composition comprises a surfactant, wherein the surfactant comprises 5-35% of the total composition; alternatively, 5-20%; alternatively, 10-20%; alternatively, 15-35%, alternatively, 15-25%; or alternatively, 17-22%.

In embodiments of the present disclosure, the pharmaceutical composition comprises a co-surfactant, wherein the co-surfactant comprises 5-35% by weight of the total composition, alternatively, 5-20%, alternatively, 10-20%, alternatively, 15-35%, alternatively, 15-25%, or alternatively, 17-22%.

In embodiments of the present disclosure, the pharmaceutical composition comprises a complete system composed of a surfactant, co-surfactant, and lipophilic phase, wherein the complete system comprises less than 45% by weight of the total composition, alternatively, 25-45%; alternatively, 31-41%; alternatively, 31-39%; alternatively, 33-37%, alternatively, about 36%; or alternatively, 36%.

In other embodiments of the present disclosure, the pharmaceutical composition comprises an antioxidant, wherein the antioxidant comprises less than 1% by weight of the total composition; alternatively, less than 0.5%; alternatively, less than 0.25%; alternatively, less than 0.10%; alternatively, about 0.006%; or alternatively, 0.006%.

The present disclosure also relates to a process for the preparation of solid pharmaceutical preparations as claimed containing enzymes or enzyme mixtures with at least lipolytic and optionally also proteolytic and/or amylolytic activity, preferably pancreatin and/or pancreatin-like mixtures of digestive enzymes. According to the present disclosure, the enzymes or enzyme mixtures are then converted into a suitable pharmaceutical form for oral administration with surfactant combination comprising
a surfactant chosen from the group consisting of polyethylene glycol fatty acid esters; polyethylene glycol glycerol fatty acid esters; polyethylene glycol alkyl ethers, polyethylene glycol sterol ethers, polyethylene glycol sorbitan fatty acid esters, sugar esters, polyoxyethylene-polyoxypropylene block copolymers, ionic surfactants and mixtures thereof;
a co-surfactant chosen from the group consisting of mono-acylglycerides, mono-ethers of glycerol, partial esters of propylenglycol, partial esters of polyglycerol, partial esters of ethyl diglycol and mixtures thereof, and, optionally
a lipophilic phase, which is represented by di- and/or triacylglycerides. and also optionally conventional, pharmaceutically compatible auxiliaries, carriers and/or excipients.

### EXAMPLES

The following examples describe different formulations of the oral pharmaceutical dosage forms of the present disclosure; the improved pharmacokinetic and efficacy measurements associated thereof; and dosing regimens for different patient populations.

### Example 1

In this Example, an embodiment of the oral pharmaceutical dosage forms is disclosed in Table 2.

TABLE 2

| Component | Percentage of Pellet (w/w) |
|---|---|
| Enzyme(s) | 40-80 |
| Surfactant | 5-35 |
| Co-Surfactant | 5-35 |
| Antioxidant | <1 |

### Example 2

In this Example, a further embodiment of the oral pharmaceutical dosage forms is disclosed in Table 3.

TABLE 3

| Component | Percentage of Pellet (w/w) |
|---|---|
| Pancrease Powder | 40-80 |
| Lauroyl Macrocolglycerides (LMG) | 5-35 |
| Macrogol 4000 | 5-35 |
| Butylhydroxyanisole or Vitamin E | <1 |

### Example 3

In this example, a digestibility trial was performed in PEI minipigs fed a high fat diet to compare the efficacy of an oral pharmaceutical dosage form of the present disclosure to an existing Creon® enteric coated formulation.

The digestibility trial was performed in six female pancreatic duct ligated and three control minipigs (Ellegaard), all chronically fitted with an ileo-caecal re-entrant fistula (titanium), although for this trial only fecal samples were collected. For surgical details see Tabeling R. (1998) Untersuchungen am pankreasligierten Schwein zu Effekten einer Enzymsubstitution auf die NOhrstoffverdaulichkeit (praecaecal/in toto). Dissertation zur Doctor Medicinae Veterinariae, Institilt für Tierernmahrung, Tieralrztliche Hochschule Hannover. All minipigs had recovered for more than six weeks after operation and all were shown by fecal chymotrypsin test to be pancreatic exocrine insufficient (PEI) before they were entered into the trial.

The influence of the two enzyme preparations on total digestibility were tested using a latin square design. Initially the pigs were fed meals containing one of the two preparations dosed at 28000 lipase U/meal. When all six pigs had been dosed with the low dose of each of the two preparations, the procedure was repeated for the higher dose (336000 lipase U/meal).

The trial for each enzyme dosage lasted for 14 days: i.e the pigs were fed the diet plus each new enzyme dosage for 9 days after, and then all feces were collected over the next 5 days. weighed and stored at 20° C.

The frozen feces from each pig were freeze dried, weighed again and milled. Aliquots of each of the 5 day milled samples (according to the daily fecal production) were then pooled and mixed together; i.e. giving one pooled sample for each pig for each dose of enzymes. From each pooled sample a Weender analysis was then performed for dry matter, crude ash, crude protein, crude fat and crude fibre. Dry matter was estimated by weight after freeze-drying followed by 8 h incubation at 103° C.; crude ash was estimated after 6 h in a muffle furnace; crude protein was determined by Kjeldahl analysis (raw protein=nitrogen×6.25); crude fat was determined gravimetrically after boiling for 30 min in conc. HCl followed by a 6 h extraction with petrol ether; crude fibre was determined after boiling for 30 min in 5% H2SO4 followed by boiling for 30 min in 5% NaOH and ashing the sediment in a muffle furnace. Cr₂O₃ was oxidized to chromate and chromium content calculated via extinction at 365 nm (spectrophotometer).

Digestibility values (coefficient of absorption) of each food component were estimated by the marker method according to the formula:

$$Digestibility ( \% ) = 100 - \begin{matrix}[ \\ [\end{matrix} \frac{\% {Cr}_{2} {O}_{3} in feed}{\% {Cr}_{2} {O}_{3} in chyme} \begin{matrix}\times \\ \end{matrix} \frac{\% nutrient in chyme}{\% nutrient in feed} \begin{matrix}\times 100 ] \\ ]\end{matrix}$$

The mini-pigs received either Creon® capsules or an oral pharmaceutical dosage form of the present disclosure in the form of pancreatin-Gelucire® according to Table 4.

TABLE 4

| API | Lipase U | Protease U | Amylase U |
|---|---|---|---|
| Creon ® low dose capsules | 28000 | 1780 | 26440 |
| Creon ® high dose capsules | 336000 | 21420 | 317470 |
| Pancreatin-Gelucire ® low dose | 28000 | 1810 | 26870 |
| capsules | | | |
| Pancreatin-Gelucire ® high dose | 336000 | 21700 | 322760 |
| capsules | | | |

The effect on the coefficient of fat absorption (CFA) is shown in Table 5.

TABLE 5

| API | CFA (%) |
|---|---|
| None (PEI animals) | 31.66 ± 13.78 |
| Creon ® low dose capsules | 52.03 ± 8.95 |
| Creon ® high dose capsules | 68.57 ± 9.79 |
| Pancreatin-Gelucire ® low dose capsules | 61.98 ± 11.56* |
| Pancreatin-Gelucire ® high dose capsules | 79.25 ± 6.99* |

In all cases, the administration of enzymes increased CFA in a dose-dependent manner relative to negative control PEI animals. However, it was surprising to note that CFA from each of the lipase doses in the pancreatin-Gelucire® formulations was significantly superior (p<0.05; paired t test) to the equivalent lipase dose in Creon® capsules (indicated by *). In fact, the effect of the Creon® high dose capsules was within the standard deviation of the pancreatin-Gelucire® low dose capsules, despite the pancreatin-Gelucire® low dose capsules containing about twelve times less lipase than the Creon® high dose capsules.

The effect on the coefficient of nitrogen absorption (CNA) is shown in Table 6.

TABLE 6

| API | CNA (%) |
|---|---|
| None | 58.54 ± 7.28 |
| Creon ® low dose capsules | 66.00 ± 8.56 |
| Creon ® high dose capsules | 84.54 ± 1.53 |
| Pancreatin-Gelucire ® low dose capsules | 72.33 ± 5.66* |
| Pancreatin-Gelucire ® high dose capsules | 83.31 ± 2.32 |

In all cases, the administration of proteolytic enzymes increased CNA in a dose-dependent manner relative to negative control animals. The pancreatin-Gelucire® low dose capsule was significantly superior (p<0.05; paired t test) to the corresponding Creon® low dose capsule (indicated by *). No statistical significance in CAN was found comparing the pancreatin-Gelucire® high dose capsule and the Creon® high dose capsule.

Overall, at the doses tested, the efficacy of pancreatin was increased by a factor of at least 2, 3, or 4 by formulating with Gelucire® rather than formulating as Creon® delayed-release capsules. In other words, one lipase unit of the pancreatin-Gelucire® formulation was functionally equivalent to equal to or greater than 4 lipase units of the Creon® delayed-release formulation. Although the inventors do not intend to be bound by theory, it is likely that the increased efficacy or enzyme efficiency is at least in part due to the immediate availability of the enzymes in the pancreatin-Gelucire® dose compared to the delayed release of Creon® capsules due to its enteric coating. It is further contemplated that the differences in efficacy are at least in part due to the improved lipase stability under acidic conditions and/or enhanced lipid solubilization and uptake of fatty acids/monoglycerides.

### Example 4

In Example 3, two formulations with different lipase dosages were tested and a two point dose response curve was generated. A statistically significant difference for the pancreatin-Gelucire® high dose capsule on CNA was not observed relative to the Creon® high dose capsule. This study was designed to investigate whether the lack of statistically significant improvement of CNA at the upper dose was because values were already approaching healthy values; and to confirm superior efficacy at lower doses. The doses tested are reported in Table 7.

TABLE 7

| API | Lipase U | Protease U | Amylase U |
|---|---|---|---|
| Creon ® low dose capsules | 24993 | 1293 | 19866 |
| Creon ® mid dose capsules | 99973 | 5174 | 79466 |
| Creon ® high dose capsules | 299980 | 15525 | 238445 |
| Pancreatin-Gelucire ® low dose | 24980 | 1444 | 25526 |
| capsules | | | |
| Pancreatin-Gelucire ® mid dose | 99989 | 5779 | 102176 |
| capsules | | | |
| Pancreatin-Gelucire ® high dose | 299968 | 17336 | 306527 |
| capsules | | | |

In addition to the treatment groups set forth in Table 7, healthy animals were used as positive controls, and PEI animals not treated with any enzyme supplements were used as negative controls (as indicated by *).

The effect on CFA is reported in Table 8.

TABLE 8

| API | CFA (%) |
|---|---|
| None (PEI animals) | 28.1 ± 10.6 |
| Creon ® low close capsules | 59.6 ± 9.0 |
| Creon ® mid dose capsules | 71.3 ± 7.8 |
| Creon ® high dose capsules | 80.7 ± 7.3 |
| Pancreatin-Gelucire ® low dose capsules | 69.4 ± 6.2* |
| Pancreatin-Gelucire ® mid dose capsules | 82.3 ± 6.1* |
| Pancreatin-Gelucire ® high dose capsules | 87.7 ± 4.9* |
| None (healthy animals) | 91.2 ± 1.5 |

All of the pancreatin-Gelucire® treatment groups were significantly superior (p<0.05 pair t test with Holm-Bonferroni correction) to the corresponding Creon® treatment groups.

The effect on fecal CNA is reported in Table 9.

TABLE 9

| API | Fecal CNA (%) |
|---|---|
| None (PEI animals) | 48.4 ± 13.0 |
| Creon ® low dose capsules | 62.5 ± 8.3 |
| Creon ® mid dose capsules | 70.0 ± 6.9 |
| Creon ® high dose capsules | 81.4 ± 3.2 |
| Pancreatin-Gelucire ® low dose capsules | 73.4 ± 2.9** |
| Pancreatin-Gelucire ® mid dose capsules | 83.7 ± 2.1** |
| Pancreatin-Gelucire ® high dose capsules | 86.7 ± 1.6* |
| None (healthy animals) | 91.7 ± 1.6 |

All of the pancreatin-Gelucire® treatment groups were significantly superior (**=p<0.01, *=p<0.05 pair t test with Holm-Bonferroni correction) to the corresponding Creon® treatment groups.

The effect on ileal CNA is reported in Table 10.

TABLE 10

| API | Ileal CNA (%) |
|---|---|
| None (PEI animals) | 32.9 ± 3.8 |
| Creon ® low dose capsules | 41.6 ± 6.8 |
| Creon ® mid dose capsules | 56.1 ± 7.3 |
| Creon ® high dose capsules | 69.4 ± 3.8 |
| Pancreatin-Gelucire ® low dose capsules | 55.1 ± 5.9** |
| Pancreatin-Gelucire ® mid dose capsules | 70.5 ± 3.8* |
| Pancreatin-Gelucire ® high dose capsules | 74.3 ± 3.4 |
| None (healthy animals) | 79.9 ± 5.9 |

Both of the lower doses of pancreatin-Gelucire® were significantly superior (**=p<0.01, *=p<0.05 pair t test with Holm-Bonferroni correction) to the corresponding Creon® treatment groups. However, a statistically significant difference was not observed at the high dose, which may be attributable to the CNA level approaching healthy animal levels.

The effect on ileal coefficient of starch absorption (CSA) is reported in Table 11.

TABLE 11

| API | Ileal CSA (%) |
|---|---|
| None (PEI animals) | 65.9 ± 20.3 |
| Creon ® low dose capsules | 57.9 ± 20.0 |
| Creon ® mid dose capsules | 84.6 ± 16.3 |
| Creon ® high dose capsules | 91.7 ± 7.1 |
| Pancreatin-Gelucire ® low dose capsules | 82.6 ± 11.8** |
| Pancreatin-Gelucire ® mid dose capsules | 93.1 ± 2.6 |
| Pancreatin-Gelucire ® high dose capsules | 95.9 ± 1.9 |
| None (healthy animals) | 98.8 ± 0.3 |

Ileal CSA was only moderately reduced in untreated PEI animals. Ileal CSA was dose dependently improved and normalized by both Creon® formulations and pancreatin-Gelucire® formulations. At the lowest dose tested, the pancreatin-Gelucire® formulation was significantly more effective (** p<0.01 paired t test with Holm-Bonferroni correction) than the lowest dose of the Creon® formulation, which was not different than untreated PET animals at this dose.

These findings confirmed and extended the findings of Example 3. One lipase unit of the pancreatin-Gelucire® formulation was estimated to be functionally equivalent to approximately 3.5 lipase units Creon® delayed-release formulation, which agrees well with the observation in Example 3 that 1 lipase unit of the pancreatin-Gelucire® formulation was equivalent to greater or equal to 4 lipase units Creon® delayed-release formulation. The pancreatin-Gelucire® formulations caused significantly higher ileal and fecal CNA than Creon®, at least at the lower doses, but statistical significance is reduced or lost at the highest dose because the slope of the dose response curve to the pancreatin-Gelucire® formulations flattens off as values approach healthy control. Considering both ileal and fecal CNA findings, 1 protease unit in the pancreatin-Gelucire® formulation was functionally equivalent to 3-4 protease units in the Creon® delayed-release formulation.

These findings show that the coefficient of starch absorption (CSA) is also significantly higher after administration of low dose pancreatin-Gelucire® capsules compared to low dose Creon® delayed-release capsules. The dose response curves were not parallel, hindering any quantitative comparison so that it can only be deduced that a pancreatin-Gelucire® formulation appears to be at least twice as effective as Creon® capsules on CSA.

### Example 5

A phase 2 clinical trial is performed investigating whether the efficacy of pancreatin can be increased or maintained through the use of the present pharmaceutical composition or oral pharmaceutical dosage form compared to Creon® capsules. Table 12 describes the pharmaceutical composition that is investigated.

TABLE 12

| Component | Percent by weight |
|---|---|
| Lipase-containing Enzyme Mixture | 59-69 |
| Lauroyl Macrogolglycerides (LMG) | 15.5-20.5 |
| Polyethylene Glycol (PEG) 4000 | 15.5-20.5 |
| Antioxidant | 0.0047-0.0062 |

The pharmaceutical composition is made by forming pellets of the foregoing components as follows. Different batches of the API (the lipase-containing enzyme mixture) are blended to produce a homogeneous mixture. The LMG, PEG 4000, and antioxidant, for example, butylhydroxyanisole, are melted in a drying cabinet and mixed with a stirrer for generating a homogenous molten mass. The temperature inside the drying cabinet is 75±5° C. and the mixing time is controlled to be not less than 30 min. The prepared API mixture and molten mass are fed into the twin screw extruder. The composition is adjusted by controlling the solid and liquid feed rates in order to produce consistent extrudates. The screw speed may be 70-80 rpm, preferably 75 rpm. The product temperature should be 108±5° C. After the extrudate is cooled to room temperature, it is spheronized in order to shape the strands into pellets. During spheronization the jacket temperature is controlled to not more than 50° C. with a setpoint of 49° C., the rotation speed of the spheronizer is 1019 rpm±5% and the run time is 8±2 min. The pellets are passed through two different sizes sieve screens to separate the pellets with the desired size. The two sieves have a mesh size of 0.7 and 1.6 mm, so the pellets have a particular size of 0.7 mm to 1.6 mm. The pellets are then put in capsules to form oral dosage forms. The pellets are filled in the capsule shells to a target fill weight of 46-85 mg for 4,000 Ph. Eur. U. and 273-507 mg for 30,000 Ph. Eur. U. The target fill weight within this range is defined based on the lipase activity of the intermediate pellets by calculating the required amount of pellets to achieve the target lipase activity per capsule. The fill weight is within ±7.5% of the target fill weight for capsule size 4 and ±5.0% of the target fill weight for capsule size 0.

Table 13 describes the total daily dose and estimated units/kilogram dose.

TABLE 13

| | Total Daily Dose | Total dose by body weight |
|---|---|---|
| API | (lipase units) | (lipase units per kg) |
| Composition of | About 30,000 | About 3 75 |
| Table 12 | (e.g., 32,000) | |
| Composition of | About 120,000 | About 1,500 |
| Table 12 | (e.g., 122,000) | |
| Composition of | About 240,000 | About 3,000 |
| Table 12 | | |
| Composition of | About 400,000 | About 6,000 |
| Table 12 | (e.g., 394,000) | |
| Commercially | About 400,000 | About 10,000 |
| available Creon | | |

The total dose by weight (units per kg) for the composition of Table 12 is reduced compared to the commercially available Creon® capsules due to the improved effectiveness and/or enzyme efficiency afforded by the composition of Table 12. In other words, more lipase units of the commercially available Creon® capsules per kilogram are required to achieve 400,000 total daily lipase units compared to the composition of Table 12.

During the double-blind treatment period, the subjects will receive a standardized diet with 100 g daily fat content and normal to low fiber content.

The following inclusion criteria are used in the clinical trial. (1) Subject is 12 years old or older at the time of the consent signature. (2) Subject has a diagnosis of CF previously confirmed by: (a) a sweat chloride test greater or equal to 60 mmol/Ls and/or (b) two CF causing CFTR mutations and CF clinical features. (3) Subject has a documented clinically confirmed diagnosis of pancreatic exocrine insufficiency. (4) Subject has human fecal elastase <100 μg/g stool at screening. (5) Subject has PET that is currently clinically controlled (no clinically overt steatorrhea or diarrhea) under treatment with a commercially available PERT, on an individually established dose regimen for more than 3 months, with a daily dose not exceeding 10,000 U lipase/kg/day.

The following exclusion criteria are used in the clinical trial. (1) Subject is <18 years of age and has a Body Mass index (BMI) Z-Score below −1.5 (minus 1.5). (2) Subject has a history of any of the following gastrointestinal disorders: (a) pancreatitis within 6 months prior to study entry: (b) fibrosing colonopathy; (c) distal ileal obstruction syndrome (DIOS) within 6 months prior to study entry; (d) celiac disease; (e) gastric bypass or partial/total gastrecetomy; (f) Crohn's disease; (g) small bowel surgery (other than minor resection due to meconium ileus without resulting in malabsorption syndrome): and (h) any type of malignancy involving the digestive tract in the last 5 years. (3) Subjects with diabetes mellitus, for which the study specific dietary requirements may not be appropriate. (4) Subject has a history or other endocrine or respiratory (except mild asthma) medical illness not related to CF, which might limit participation in or completion of the study. (5) Subjects requiring Neo-gastric, G-tubes, or J-tubes. (6) Subject is currently participating in any other interventional clinical study or has taken any experimental drug within 30 days prior to screening. (7) Subject is known to be HIV positive. (8) Subject has a history of allergic reaction sensitivity or hypersensitivity to pancreatin or inactive ingredients (excipients) of delayed release Creon or the contents listed in Table 7.

The total daily dose will be administered orally, divided proportionally based on three meals and two snacks per day. For example, one-fourth of the total daily dose may be administered in each of the three main meals and one-eighth of the total daily dose may be administered in each of two daily snacks.

One or more of the compositions containing a lower lipase dose is expected to result in equal or better efficacy compared to the Creon® capsules group. Further, one or more of the groups of the composition of Table 13 with 32,000, 122,000, 240,000, and 390,000 total daily FIP units of lipase will result in >80% or >85% CFA.

The following efficacy variables will be studied: CFA, CAN, stool fat content and stool weight. The following safety variables will be studied: clinical symptomotology associated with PEI (stool frequency, stool consistency, abdominal pain, flatulence), vital signs, physical examination findings, safety laboratory values and adverse events.

### Example 6

Dosing regimens involving the administration of lipase to PEI subjects are dependent on the age of the patient. Prior lipase compositions have been administered according to particular guidelines for treatment of infants that vary from the guidelines for treating children aged 1-4 and adults/children aged 4 and above. In infants, the guidelines stated that dosing of prior lipase compositions should be initiated at a dose of 2,000 to 5,000 lipase units for each feeding (typically 120 ml) and adjusted up to a dose no greater than 2,500 lipase units per kilogram of body weight per feeding with a maximum daily dose of 10,000 lipase units per kilogram per day. The unique composition of Table 11 is dosed in the same or similar timing as described for the prior lipase treatment. However, the lipase dose is decreased in the composition of Table 12 relative to existing compositions without compromising the level of effectiveness. Table 14 lists the prior lipase dose for treating infants and equivalent or superior doses in different embodiments of the present methods.

TABLE 14

| Prior Lipase | | | | | |
|---|---|---|---|---|---|
| Compositions | Embodiment A | Embodiment B | Embodiment C | Embodiment D | Embodiment E |
| (lipase | (lipase | (lipase | (lipase | (lipase | (lipase |
| U/meal) | U/meal) | U/meal) | U/meal) | U/meal) | U/meal) |
| 2,000 | 500 | 667 | 1,000 | 1,333 | 1,538 |
| 5,000 | 1,250 | 1,667 | 2,500 | 3,333 | 3,846 |

As noted elsewhere, the dose of lipase units per snack would be half of the dose of lipase units per meal. As would be appreciated by one skilled in the art, the lipase compositions of the present disclosure allows the lipase dose in the treatment of infants to be lowered, relative to existing lipase compositions, by a factor of at least 4; alternatively, by a factor of at least 3; alternatively, by a factor of at least 2; alternatively, by a factor of at least 1.5; or alternatively, by a factor of at least 1.3.

In one embodiment, the lipase compositions of the present disclosure are administered to infants at a lipase dose ranging from approximately 500 to approximately 4,000 lipase [PhEur] units per meal. In another embodiment, the lipase compositions of the present disclosure are administered to infants at a lipase dose ranging from approximately 800 to approximately 2,500 lipase [PhEur] units per meal.

In addition to the dose amounts set forth in Table 14, it is contemplated that maximum lipase dose for the treatment of infants will be established for the lipase compositions of the present disclosure. Table 15 lists the maximum doses of prior lipase compositions compared to the comparable or superior maximum doses of the lipase compositions of the present disclosure.

TABLE 15

| | Prior Lipase | Embodiment | Embodiment | Embodiment | Embodiment | Embodiment |
|---|---|---|---|---|---|---|
| | Compositions | A | B | C | D | E |
| Max. lipase | 2,500 | 625 | 833 | 1,250 | 1,667 | 1,923 |
| U/kg body | | | | | | |
| weight/meal | | | | | | |
| Max. lipase | 10,000 | 2,500 | 3,333 | 5,000 | 6,667 | 7,692 |
| U/kg | | | | | | |
| bodyweight/day | | | | | | |

The present methods using the lipase compositions described herein allow the maximum lipase dose in the treatment of infants to be lowered, relative to existing methods with existing lipase compositions, by a factor of 4; alternatively, by a factor of 3; alternatively, by a factor of 2; alternatively, by a factor of 1.5; or alternatively, by a factor of 1.3.

In some embodiments, the present methods comprise administering lipase compositions of the present disclosure to infants in a manner not to exceed approximately 2,000; alternatively, approximately 1,700; or alternatively, approximately 1,300 lipase [PhEur] units per meal. In other embodiments, the lipase compositions of the present disclosure are administered to infants in a manner not to exceed approximately 8,000; alternatively, approximately 7,000; or alternatively, approximately 5,000 lipase [PhEur] units per kilogram bodyweight per day.

The infant doses described in Tables 14 and 15 are particularly preferred for infants suffering from cystic fibrosis.

### Example 7

Dosing regimens and methods involving lipase for the treatment of children and adults vary from the dosing regimens and methods for the treatment of infants. However, the methods using the lipase compositions of the present disclosure are still consistently equal or superior to methods using prior lipase compositions. Prior lipase compositions are dosed an initial dose of 1,000 lipase units/kg bodyweight/meal for children less than four years of age and with 500 lipase units/kg bodyweight/meal for those over the age of four. Most patients should not exceed 10,000 lipase units/kg bodyweight/day or 4,000 lipase units per gram fat intake. Use of the lipase compositions of the present disclosure can lower both the initial dose and the maximum doses in the treatment of disease in children and adults. For example, Table 16 depicts the initial doses for the prior lipase compositions compared to the different doses of the present lipid composition, wherein the efficacy observed with the prior lipase composition dose is not lost despite the lower dose of the present lipid composition.

TABLE 16

| Prior Lipase | | | | | |
|---|---|---|---|---|---|
| Compositions | Embodiment A | Embodiment B | Embodiment C | Embodiment D | Embodiment E |
| (lipase | (lipase | (lipase | (lipase | (lipase | (lipase |
| U/kg/meal) | U/kg/meal) | U/kg/meal) | U/kg/meal) | U/kg/meal) | U/kg/meal) |
| 1,000 | 250 | 333 | 500 | 667 | 769 |
| 500 | 125 | 167 | 250 | 333 | 385 |

As noted elsewhere, the amount of lipase units per snack would be half of the amount of lipase units per meal. The present methods using the lipase compositions described herein allow the lipase dose in the treatment of infants to be lowered, relative to existing methods with existing lipase compositions, by a factor of 4; alternatively, by a factor of 3; alternatively, by a factor of 2; alternatively, by a factor of 1.5; or alternatively, by a factor of 1.3.

In some embodiments, the methods comprise administering an initial dose of the lipase composition which is less than 500 lipase units/kg/meal. In other embodiments, the initial dose of the lipase composition of the present disclosure ranges between 250 and 800, or alternatively, 300 and 500 U lipase/kg body weight/meal for children less than 4 years of age. In another embodiment, the initial dose of the lipase composition of the present disclosure ranges between 125 and 400, or alternatively, 170-350 U lipase/kg/meal for patients aged four and above.

In addition to the dosing amounts set forth in Table 16, it is contemplated that maximum lipase dose for the treatment of children and adults will be established for the lipase compositions of the present disclosure. Table 17 lists the maximum doses of prior lipase compositions compared to the comparable or superior maximum doses of the lipase compositions of the present disclosure.

TABLE 17

| | Prior Lipase | Embodiment | Embodiment | Embodiment | Embodiment | Embodiment |
|---|---|---|---|---|---|---|
| | Compositions | A | B | C | D | E |
| Max. lipase | 4,000 | 1,000 | 1,333 | 2,000 | 2,667 | 3,077 |
| U/g/fat intake | | | | | | |
| Max. lipase | 10,000 | 2,500 | 3,333 | 5,000 | 6,667 | 7,692 |
| U/kg | | | | | | |
| bodyweight/day | | | | | | |

The present methods using the lipase compositions described herein allow the maximum lipase dose in the treatment of children and adults to be lowered, relative to existing methods using existing lipase compositions, by a factor of 4; alternatively, by a factor of 3; alternatively, by a factor of 2; alternatively, by a factor of 1.5; or alternatively, by a factor of 1.3.

In some embodiments, the present methods comprise administering lipase compositions of the present disclosure to children and adults in a manner not to exceed approximately 4,000; alternatively, approximately 3,000; or alternatively, approximately 2.000 lipase [PhEur] units per gram of fat intake per day. In another embodiment, the lipase compositions of the present disclosure are administered to children and adults in a manner not to exceed approximately 8,000; alternatively, approximately 7000. or alternatively, approximately 5,000 lipase [PhEur] units per kilogram bodyweight per day.

The children and adult doses described in Tables 16 and 17 are particularly preferred for children and adults suffering from cystic fibrosis.

### Example 8

For patients suffering from maldigestion not caused by cystic fibrosis, other dosing regimens may be appropriate. Prior lipase compositions are dosed to individuals suffering from maldigestion based on the degree of the maldigestion and the fat content of the individual's typical diet. The dosing range of the prior lipase compositions for this patient population ranges from 25,000 to 80,000 lipase units per meal. Use of the lipase compositions of the present disclosure shifts this range of efficacious doses lower. For example, Table 17 depicts the lowest and highest doses for the prior lipase compositions compared to the different doses of the present lipid composition, wherein the efficacy observed with the prior lipase composition dose is not lost despite the lower dose of the present lipid composition.

TABLE 17

| Prior Lipase | | | | | |
|---|---|---|---|---|---|
| Compositions | Embodiment A | Embodiment B | Embodiment C | Embodiment D | Embodiment E |
| (lipase | (lipase | (lipase | (lipase | (lipase | (lipase |
| U/kg/meal) | U/kg/meal) | U/kg/meal) | U/kg/meal) | U/kg/meal) | U/kg/meal) |
| 25,000 | 6,250 | 8,333 | 12,500 | 16,667 | 19,231 |
| 80,000 | 20,000 | 26,667 | 40,000 | 53,333 | 61,538 |

As noted elsewhere, the amount of lipase units per snack would be half of the amount of lipase units per meal. As would be appreciated by one skilled in the art, the lipase compositions of the present disclosure allows the lipase dose in the treatment of patients suffering from maldigestion to be lowered, relative to existing lipase compositions, by a factor of 4; alternatively, by a factor of 3; alternatively, by a factor of 2; alternatively, by a factor of 1.5; or alternatively, by a factor of 1.3.

In one embodiment, the dose of the lipase composition of the present disclosure ranges between 6,250 and 65,000, or alternatively, 8,000 and 40,000 lipase units/meal for patients suffering from maldigestion.

Some exemplary embodiments of the present methods are set forth below:

1. A method of treating exocrine pancreatic insufficiency in a subject in need of treatment, the method comprising:

administering a total daily dose of an enzyme or an enzyme mixture with at least lipolytic activity which exerts its action in the gastrointestinal tract, wherein the total daily dose has lipolytic activity that is equal to about 240,000 FIP units or less, alternatively 122,000 FIP units or less, alternatively about 120,000 FIP units or less, alternatively 32,000 FIP units or less, alternatively about 30,000 FIP units or less, wherein the total daily lipase dose is sufficient to treat exocrine pancreatic insufficiency, and

the enzyme or enzyme mixture is in one or more pharmaceutical compositions which does not contain an enteric coating.

2. The method of embodiment 1, wherein the subject has exocrine pancreatic insufficiency associated with or due to cystic fibrosis, chronic pancreatitis, or pancreatectomy.

3. The method of embodiment 1 or 2, wherein total daily dose is sufficient to raise the coefficient of fat absorption (CFA) of the subject to about 80% or greater, alternatively about 85% percent or greater.

4. The method of any of embodiments 1, 2 or 3, wherein the pharmaceutical compositions comprise a surfactant system comprising (a) a surfactant in an amount of 2% to 90% by weight of the surfactant system selected from polyethylene glycol fatty acid mono-esters and/or di-esters with aliphatic C₆-C₂₂ carboxylic acids; polyethylene glycol glycerol fatty acid esters with aliphatic C₆-C₂₂ carboxylic acids; polyethylene glycol alkyl mono-ethers and/or di-ethers with aliphatic C₁₂-C₁₈ alcohols, and mixtures of the foregoing; (b) a co-surfactant in an amount of 5% to 60% by weight of the surfactant system, selected from a group consisting of monoacylglycerides with aliphatic C₆-C₂₂ carboxylic acids, monoethers of glycerol ethers with aliphatic C₁₂-C₁₈ alcohols, partial esters of propylenglycol with aliphatic CC₆-C₂₂ carboxylic acids, partial esters of polyglycerol with aliphatic C₆-C₂₂ carboxylic acids, and mixtures of the foregoing; and (c) a lipophilic phase in an amount of 0% to 70% by weight of the surfactant system, represented by diacylglyerides and/or triacylglycerides with aliphatic C₆-C₂₂ carboxylic acids; wherein the surfactant system comprises 10% to 95% by weight of the pharmaceutical composition.

5. The method of embodiment 4, wherein the pharmaceutical compositions further comprise one or more polyethylene glycols having an average molecular weight of about 200 to about 6000, alternatively polyethylene glycol 4000.

6. A method of treating diseases responsive to pancreatin enzyme replacement therapy comprising:

administering a dose of a pharmaceutical composition to a patient in need thereof,

wherein the pharmaceutical composition comprises:
an enzyme or enzyme mixture with at least lipolytic activity and at least one surfactant,

wherein the lipase dose of the pharmaceutical composition is less than about 300 U lipase/kg body weight/meal or less, alternatively 320 U lipase/kg body weight/meal.

7. The method of embodiment 6, wherein the administering occurs whenever the patient consumes a snack or a meal.

8. The method of embodiment 6 or 7, wherein the pharmaceutical composition further comprises at least one antioxidant, at least one co-surfactant, or a combination thereof.

9. The method of any of embodiments 6, 7 or 8, wherein the pharmaceutical composition is capable of releasing at least 85% of the lipase contained therein within one hour of contacting an in vivo pH equal or greater to 6.0.

10. A pharmaceutical composition comprising:

an enzyme or an enzyme mixture with at least lipolytic activity which exerts its action in the gastrointestinal tract,

at least one surfactant,

at least one co-surfactant,

wherein the pharmaceutical composition is capable of releasing at least 85% of the lipase contained therein within one hour of contacting an in vivo pH equal or greater to 6.0.

11. The pharmaceutical composition of embodiment 10, further comprises at least one anti-oxidant.

12. A pharmaceutical composition comprising:

an enzyme or an enzyme mixture with at least lipolytic activity which exerts its action in the gastrointestinal tract,

at least one surfactant, and

at least one co-surfactant,

wherein the lipase is present in an amount equal to about 4,000 FIP units or about 30,000 FIP units, and

wherein the pharmaceutical composition does not contain an enteric coating.

13. The pharmaceutical composition of embodiment 12, further comprising at least one anti-oxidant.

14. A pharmaceutical composition or oral pharmaceutical dosage form comprising:

an enzyme or an enzyme mixture with at least lipolytic activity which exerts its action in the gastrointestinal tract,

at least one surfactant, and

at least one co-surfactant,

wherein the amount of the enzyme or enzyme mixture is greater than 60% by weight the total composition.

15. The pharmaceutical composition of embodiment 14, further comprises at least one anti-oxidant.

16. An oral pharmaceutical dosage form comprising:

an enzyme or an enzyme mixture with at least lipolytic activity which exerts its action in the gastrointestinal tract, and

at least one surfactant.

wherein the oral pharmaceutical dosage form releases at least 85% of the enzyme or an enzyme mixture with at least lipolytic activity contained therein within one hour of contacting an in vivo pH equal or greater to 6.0.

17. The pharmaceutical composition of embodiment 16, further comprises at least one anti-oxidant, at least one co-surfactant, or a combination thereof.

18. A method of treating exocrine pancreatic insufficiency, comprising:

administering a dose of a pharmaceutical composition or oral pharmaceutical dosage form of any preceding claim to a patient in need thereof;

wherein the dose of the pharmaceutical composition or oral pharmaceutical dosage form comprises less than about 6,000 lipase units/kg.

19. A method of treating a subject in need of treatment for exocrine pancreatic insufficiency with a lower dose of an enzyme or enzyme mixture, the method comprising:

administering a first pharmaceutical composition comprising an enzyme or enzyme mixture having at least lipolytic activity to a subject having exocrine pancreatic insufficiency, wherein the first pharmaceutical composition does not contain an enteric coating, wherein the first pharmaceutical composition provides increased fat absorption as compared to a second pharmaceutical composition that contains an enteric coating and comprises substantially the same dose of the enzyme or enzyme mixture with at least lipolytic activity as the first pharmaceutical composition.

20. The method of embodiment 19, wherein the subject experiences a coefficient of fat absorption (CFA) of about 80% or greater, alternatively about 85% percent or greater due to the administration of the first pharmaceutical composition.

21. The method of embodiment 19 or 20, wherein the first pharmaceutical composition comprises (a) a surfactant in an amount of 2% to 90% by weight of the surfactant system selected from polyethylene glycol fatty acid mono-esters and/or di-esters with aliphatic C₆-C₂₂ carboxylic acids; polyethylene glycol glycerol fatty acid esters with aliphatic C₆-C₂₂ carboxylic acids; polyethylene glycol alkyl mono-ethers and/or di-ethers with aliphatic C₁₂-C₁₈ alcohols, and mixtures of the foregoing; (b) a co-surfactant in an amount of 5% to 60% by weight of the surfactant system, selected from a group consisting of monoacylglycerides with aliphatic C₆-C₂₂ carboxylic acids, monoethers of glycerol ethers with aliphatic C₁₂-C₁₈ alcohols, partial esters of propylenglycol with aliphatic CC₆-C₂₂ carboxylic acids, partial esters of polyglycerol with aliphatic C₆-C₂₂ carboxylic acids, and mixtures of the foregoing; and (c) a lipophilic phase in an amount of 0% to 70% by weight of the surfactant system, represented by diacylglyerides and/or triacylglycerides with aliphatic C₆-C₂₂ carboxylic acids; wherein the surfactant system comprises 10% to 95% by weight of the pharmaceutical composition.

1. A method of treating exocrine pancreatic insufficiency in a subject in need of treatment, the method comprising:
administering a dose of an enzyme or an enzyme mixture with at least lipolytic activity which exerts its action in the gastrointestinal tract,
wherein the enzyme or enzyme mixture is one or more pharmaceutical compositions which does not contain an enteric coating.

2. The method of claim 1, wherein the subject has exocrine pancreatic insufficiency associated with, due to, or caused by cystic fibrosis, chronic pancreatitis, or pancreatectomy.

3. The method of claim 1, wherein the dose is a total daily dose, wherein the total daily dose of the enzyme or mixture with lipolytic activity is approximately 240,000 lipase units or less; alternatively, equal to approximately 122,000 lipase units or less; alternatively, equal to approximately 120,000 lipase units or less; alternatively, equal to approximately 32,000 lipase units or less; alternatively, equal to approximately 30,000 lipase units or less.

4. The method of claim 1, wherein the dose is a maximum total daily dose, wherein the maximum total daily dose per kilogram of the enzyme or mixture with lipolytic activity is less than approximately 10,000 lipase units per kilogram; alternatively, less than approximately 2500 lipase units per kilogram; alternatively, less than approximately 3333 lipase units per kilogram; alternatively, less than approximately 5000 lipase units per kilogram; alternatively, less than approximately 6667 lipase units per kilogram; or alternatively, less than approximately 7692 lipase units per kilogram.

5. The method of claim 1, wherein the dose is a dose per meal, wherein the dose per meal of the enzyme or mixture with lipolytic activity is less than approximately 500 lipase units per kilogram body weight; alternatively, less than approximately 320 lipase units per kilogram body weight; alternatively, less than approximately 500 lipase units per kilogram body weight; alternatively, equal to approximately 125 lipase units per kilogram body weight; alternatively, equal to approximately 167 lipase units per kilogram body weight; alternatively, equal to approximately 250 lipase units per kilogram body weight; alternatively, equal to approximately 333 lipase units per kilogram body weight; or alternatively, equal to approximately 385 lipase units per kilogram body weight.

6. The method of claim 1, wherein the dose is a maximum dose per gram of fat intake, wherein the maximum dose per gram of fat intake of the enzyme or mixture with lipolytic activity is less than approximately 4000 lipase units per kilogram; alternatively, less than or equal to approximately 1000 lipase units per kilogram; alternatively, less than or equal to approximately 1333 lipase units per kilogram; alternatively, less than or equal to approximately 2000 lipase units per kilogram; alternatively, less than or equal to approximately 2667 lipase units per kilogram; or alternatively, less than or equal to approximately 3077 lipase units per kilogram.

7. The method of claim 1, wherein the subject is an adult or child aged four years or older.

8. The method of claim 1, wherein the subject is an infant, wherein the dose is a maximum dose per meal, wherein the maximum dose per meal of the enzyme or mixture with lipolytic activity is less than approximately 2500 lipase units per kilogram; alternatively, less than or equal to approximately 625 lipase units per kilogram; alternatively, less than or equal to approximately 833 lipase units per kilogram; alternatively, less than or equal to approximately 1250 lipase units per kilogram; alternatively, less than or equal to approximately 1667 lipase units per kilogram; or alternatively, less than or equal to approximately 1923 lipase units per kilogram.

9. The method of claim 1, wherein the subject is an infant, wherein the dose is a dose per meal, wherein the dose per meal of the enzyme or mixture with lipolytic activity is less than approximately 2000 lipase units per kilogram; alternatively, equal to approximately 500 lipase units per kilogram; alternatively, equal to approximately 667 lipase units per kilogram; alternatively, equal to approximately 1000 lipase units per kilogram; alternatively, equal to approximately 1333 lipase units per kilogram; or alternatively, equal to approximately 1538 lipase units per kilogram.

10. The method of claim 1, wherein the subject is an infant, wherein the dose is a maximum dose per kilogram bodyweight per day, wherein the maximum dose per kilogram bodyweight per day of the enzyme or mixture with lipolytic activity is less than approximately 10,000 lipase units per kilogram; alternatively, less than or equal to approximately 2500 lipase units per kilogram; alternatively, less than or equal to approximately 3333 lipase units per kilogram; alternatively, less than or equal to approximately 5000 lipase units per kilogram; alternatively, less than or equal to approximately 6667 lipase units per kilogram; or alternatively, less than or equal to approximately 7692 lipase units per kilogram.

11. The method of claim 1, wherein the subject is a child younger than four years old, wherein the dose is a dose per meal, wherein the dose per meal of the enzyme or mixture with lipolytic activity is less than approximately 1000 lipase units per kilogram; alternatively, equal to approximately 250 lipase units per kilogram; alternatively, equal to approximately 333 lipase units per kilogram; alternatively, equal to approximately 500 lipase units per kilogram; alternatively, equal to approximately 667 lipase units per kilogram; or alternatively, equal to approximately 769 lipase units per kilogram.

12. The method of claim 1, wherein the dose is sufficient to raise the coefficient of fat absorption (CFA) of the subject to about 80% or greater, alternatively about 85% percent or greater.

13. The method of claim 1, wherein at least 85% of the enzyme or mixture contained in the pharmaceutical composition is released within one hour of contacting an in vivo pH equal or greater to 6.0.

14. The method of claim 1, wherein the subject had previously received enzymatic replacement therapy.

15. A method of lowering a dose of enzyme or enzyme mixture having at least lipolytic activity administered to a subject in need of treatment for PEI, the method comprising:
identifying a subject in need of treatment for PEI being treating with a first dose of an enzyme or mixture having at least lipolytic activity; and
administering a pharmaceutical composition comprising an enzyme or enzyme mixture having at least lipolytic activity at a second dose to the subject;
wherein the second dose is more than 10% less than the first dose, without a significant decrease in fat absorption by the subject.

16. The method of claim 15, wherein the subject has an increase in fat absorption when taking the second dose of the enzyme or mixture compared to fat absorption when taking the first dose.

17. The method of claim 16, wherein the second dose is sufficient to raise the coefficient of fat absorption (CFA) of the subject to about 80% or greater, alternatively about 85% percent or greater.

18. The method of claim 15, wherein the pharmaceutical composition further comprises at least one surfactant, at least one antioxidant, at least one co-surfactant, or a combination thereof.

19. The method of claim 15, wherein the pharmaceutical composition comprise a surfactant system comprising (a) a surfactant in an amount of 2% to 90% by weight of the surfactant system selected from polyethylene glycol fatty acid mono-esters and/or di-esters with aliphatic C₆-C₂₂ carboxylic acids; polyethylene glycol glycerol fatty acid esters with aliphatic C₆-C₂₂ carboxylic acids; polyethylene glycol alkyl mono-ethers and/or di-ethers with aliphatic C₁₂-C₁₈ alcohols, and mixtures of the foregoing; (b) a co-surfactant in an amount of 5% to 60% by weight of the surfactant system, selected from a group consisting of monoacylglycerides with aliphatic C₆-C₂₂ carboxylic acids, monoethers of glycerol ethers with aliphatic C₁₂-C₁₈ alcohols, partial esters of propylenglycol with aliphatic CC₆-C₂₂ carboxylic acids, partial esters of polyglycerol with aliphatic C₆-C₂₂ carboxylic acids, and mixtures of the foregoing; and (c) a lipophilic phase in an amount of 0% to 70% by weight of the surfactant system, represented by diacylglyerides and/or triacylglycerides with aliphatic C₆-C₂₂ carboxylic acids; wherein the surfactant system comprises 10% to 95% by weight of the pharmaceutical composition.

20. The method of claim 19, wherein the pharmaceutical compositions further comprise one or more polyethylene glycols having an average molecular weight of about 200 to about 6000, alternatively polyethylene glycol having an average molecular weight of 4000.
